(19) United States
(12) Patent Application Publication
(10) Pub. No.: US 2023/0165968 A1
(43) Pub. Date: Jun. 1, 2023

(54) CANCER TREATMENT METHODS

(71) Applicant: BOLT BIOTHERAPEUTICS, INC., Redwood City, CA (US)

(72) Inventors: Shelley Erin ACKERMAN, Redwood City, CA (US); Michael N. ALONSO, Redwood City, CA (US); David DORNAN, Redwood City, CA (US); Marcin L. KOWANETZ, Redwood City, CA (US); Robert S. SIKORSKI, Redwood City, CA (US); Steven A. SMITH, Redwood City, CA (US)

(73) Assignee: BOLT BIOTHERAPEUTICS, INC., Redwood City, CA (US)

(21) Appl. No.: 17/802,392

(22) PCT Filed: Feb. 25, 2021
(86) PCT No.: PCT/US2021/019667

§ 371 Date: Aug. 25, 2022

Related U.S. Application Data
(60) Provisional application No. 62/981,355, filed on Feb. 25, 2020.
(60) Provisional application No. 63/105,104, filed on Oct. 23, 2020.

(51) Int. Cl. A61K 47/68; A61P 35/00

(52) U.S. Cl. CPC A61K 47/6803; A61K 47/6855; A61P 35/00

(57) ABSTRACT

The invention provides methods for treating cancer in a subject comprising administering an immunoconjugate of formula: Ab-[TA]r or a pharmaceutically acceptable salt thereof, wherein “Ab” is an antibody construct that has an antigen binding domain that binds human epidermal growth factor receptor type 2 (HER2) and “TA” is a therapeutic agent of formula:

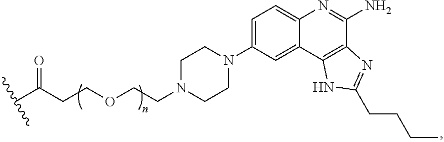

wherein n is from about 2 to about 25 and r is an average therapeutic agent to antibody ratio from 1 to 10, to a subject having cancer.

### CROSS-REFERENCE TO RELATED APPLICATIONS

This patent application claims the benefit of U.S. Provisional Patent Application No. 62/981,355, filed Feb. 25, 2020, and U.S. Provisional Patent Application No. 63/105,104, filed Oct. 23, 2020, which are incorporated by reference in their entirety herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: one 55,712 Byte ASCII (Text) file named “752185_ST25.txt,” created Feb. 5, 2021.

### BACKGROUND OF THE INVENTION

Unfortunately, the immune system is often not capable of controlling the growth and spread of cancer and other diseases and conditions. Antibodies and immune therapeutic agents have been shown to be effective treatments that assist the immune system in cancer and disease control. The simultaneous delivery of anti-tumor antibodies and therapeutic agents can be effective to treat tumors and to expand treatment options for cancer patients and other subjects. In addition, the simultaneous delivery of antibodies and therapeutic agents (i.e., immune agonists or immune antagonists) can be effective to treat diseases, conditions, and disorders, such as infections caused by viruses, bacteria, or parasites, and autoimmune diseases.

One way to simultaneously deliver antibodies and immune therapeutic agents is by conjugating the antibodies and therapeutic agents to form immunoconjugates. However, the absorption and/or metabolism rates of the antibodies and immune therapeutic agents are affected by the dosing regimen of the immunoconjugates, thereby affecting the achievement of desirable pharmacokinetic properties. Accordingly, there is a need for dosing regimens of immunoconjugates containing antibodies and therapeutic agents that provide desirable pharmacokinetic properties with respect to the treatment of diseases, conditions, and disorders. The invention provides such dosing regimens.

### BRIEF SUMMARY OF THE INVENTION

The invention provides methods for treating cancer in a subject comprising administering from about 0.01 to about 100 mg/kg of an immunoconjugate of formula: Ab-[TA]ᵣ or a pharmaceutically acceptable salt thereof, wherein “Ab” is an antibody construct that has an antigen binding domain that binds human epidermal growth factor receptor type 2 (HER2) and “TA” is a therapeutic agent of formula:

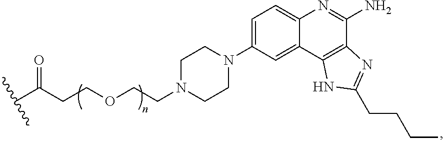

wherein n is from about 2 to about 25 and r is an average therapeutic agent to antibody ratio from 1 to 10, to a subject having cancer.

The invention also provides a method for treating cancer in a subject comprising administering from about every 3 to about every 45 days, e.g., from about every 3 to about every 35 days, an immunoconjugate of formula: Ab-[TA]ᵣ or a pharmaceutically acceptable salt thereof, wherein “Ab” is an antibody construct that has an antigen binding domain that binds HER2 and “TA” is a therapeutic agent of formula:

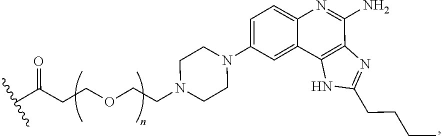

wherein n is from about 2 to about 25 and r is an average therapeutic agent to antibody ratio from 1 to 10, to a subject having cancer.

The invention further provides methods for treating cancer in a subject comprising administering from about 0.01 to about 100 mg/kg of an immunoconjugate of formula: Ab-[TA]ᵣ or a pharmaceutically acceptable salt thereof in combination with an IgG1 or IgG4 antibody to the subject, wherein the antibody is an anti-programmed cell death protein 1 (PD-1) or an anti-programmed death-ligand 1 (PD-L1) antibody.

### BRIEF DESCRIPTION OF THE SEVERAL VIEWS OF THE DRAWINGS

FIG. 1 depicts an exemplary therapy scheme used to evaluate preliminary antitumor activity of BDC-1001 as a monotherapy (Parts 1 and 3) and in combination with pembrolizumab (Parts 2 and 4) in subjects with advanced solid tumors, including subjects with advanced HER2 expressing or HER2-amplified solid tumors.

FIG. 2 is an illustration depicting a possible mechanism of action for BDC-1001. BDC-1001 may bind HER2 expressing tumor cells via the antibody variable region. Subsequently, myeloid cells bind to the Fc portion of the BDC-1001 through their Fc receptors leading to phagocytosis of the tumor cell/BDC-1001 complex. The immune-stimulating TLR7/8 agonist attached to BDC-1001 activates myeloid antigen presenting cells (APC)s such as macrophages and dendritic cells which may lead to increased cytotoxicity, processing, and presentation of tumor neoantigens that subsequently stimulate T cell-mediated anti-tumor immune response.

FIGS. 3A-3I are graphs showing that BDC-1001 (closed squares) elicits enhanced myeloid activation as defined by increased expression of CD40 (FIGS. 3A, 3D, and 3G), CD86 (FIGS. 3B, 3E, and 3H), and TNFα (FIGS. 3C, 3F, and 3I) relative to (a) trastuzumab (closed circles) and (b) the mixture of trastuzumab and the molar equivalent of therapeutic agent (closed triangles, “Trastuzumab+A103”) in HCC1954 (FIGS. 3A-C), JIMT-1 (FIGS. 3D-3F), and COLO205 (FIGS. 3G-3I) tumor models.

FIGS. 4A-4C are graphs showing that BB125 (closed squares), a trastuzumab biosimilar covalently attached to a murine TLR7 agonist via a non-cleavable linker, was significantly more effective at eliciting anti-tumor efficacy than (a) trastuzumab (closed circles) and (b) BB67 (an isotype control, closed triangles), a rituximab-TLR7 agonist conjugate, in JIMT-1 (FIG. 4A), HCC1954 1 (FIG. 4B), and COLO205 1 (FIG. 4C) tumor models.

FIGS. 5A-5B are graphs showing the results of a PK assessment wherein cynomolgus macaques were administered 2 doses of BDC-1001, 10 mg/kg (open circles) and 30 mg/kg (open squares). The PK of BDC-1001 was compared to trastuzumab (closed circles) administered intravenously 2 weeks apart at 10 mg/kg. The PK was assessed in separate assays measuring either the quantity of immunoconjugate (FIG. 5B) or the total antibody (FIG. 5A).

FIG. 6 is a graph showing the ability of BDC-1001 to activate leukocytes from cynomolgus monkey (closed squares), humans (closed circles), mice (upward pointing closed triangles), and rats (downward facing closed triangles).

FIG. 7 is a set of images from computerized tomography (CT) scans from a patient with colon cancer and lung metastases. The top images show the comparison of three distinct tumor lesions pre-treatment (left) and post 2 cycles of BDC-1001 (right). The images on the bottom show an additional distinct tumor lesion pre-treatment (left) and post 2 cycles of BDC-1001 (right). The arrows in the images are pointing to the tumor lesions.

FIG. 8 represents a schematic of the trial design to evaluate safety, pharmacokinetics, pharmacodynamics, and preliminary antitumor activity of BDC-1001 as a monotherapy (Parts 1 and 3) in subjects with advanced solid tumors. Eligibility: HER2 expressing tumors, as defined by HER2+(IHC3+ or gene amplified); HER2 Low (IHC2+ without gene amplification).

FIG. 9 represents a schematic of the trial design to evaluate safety, pharmacokinetics, pharmacodynamics, and preliminary antitumor activity of BDC-1001 in combination with immune checkpoint inhibitors (Parts 2 and 4) in subjects with advanced solid tumors. Eligibility: HER2 expressing tumors, as defined by HER2+(IHC3+ or gene amplified); HER2 Low (IHC2+ without gene amplification).

### DETAILED DESCRIPTION OF THE INVENTION

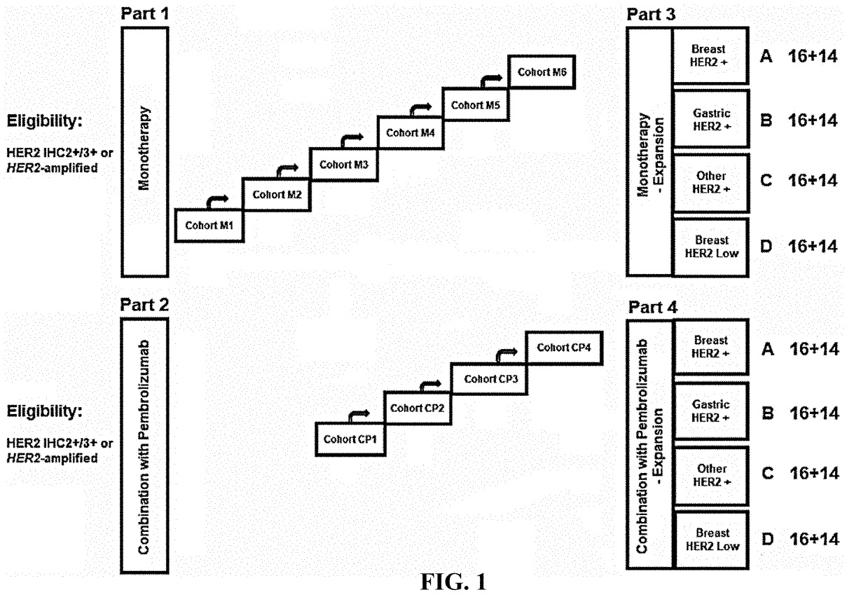

The invention provides methods for methods for treating cancer in a subject comprising administering an immunoconjugate of formula: Ab-[TA]ᵣ or a pharmaceutically acceptable salt thereof, wherein “Ab” is an antibody construct that has an antigen binding domain that binds human epidermal growth factor receptor type 2 (HER2) and “TA” is a therapeutic agent of formula:

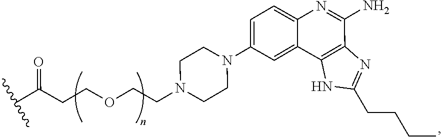

wherein n is from about 2 to about 25 and r is an average therapeutic agent to antibody ratio from 1 to 10, to a subject having cancer. The dosing regimens described herein (see FIG. 1) produce desirable treatment outcomes and pharmacokinetic (PK) properties in subjects. Additional embodiments and benefits of the inventive methods will be apparent from the description herein.

### Definitions

As used herein, the term “immunoconjugate” refers to an antibody construct that is covalently bonded to a therapeutic agent described herein.

As used herein, the phrase “therapeutic agent” refers to a chemical moiety of formula:

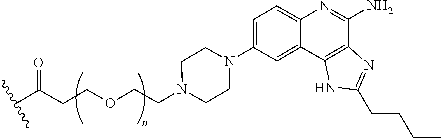

wherein n is from about 2 to about 25, as described herein. The therapeutic agent can elicit the immune response (i.e., stimulation or suppression) while bonded to the antibody construct or after cleavage (e.g., enzymatic cleavage) from the antibody construct following administration of an immunoconjugate to the subject. The therapeutic agent can be cleaved at any location such that any component (i.e., active species) of the therapeutic agent can elicit the immune response (i.e., stimulation or suppression) following administration of an immunoconjugate to the subject. The therapeutic agent can be an immune agonist or antagonist.

As used herein, the phrase “antibody construct” refers to an antibody or a fusion protein comprising (i) an antigen binding domain and (ii) an Fc domain.

As used herein, the term “antibody” refers to a polypeptide comprising an antigen binding region (including the complementarity determining region (CDRs)) from an immunoglobulin gene or fragments thereof that specifically binds and recognizes an antigen.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa) connected by disulfide bonds. Each chain is composed of structural domains, which are referred to as immunoglobulin domains. These domains are classified into different categories by size and function, e.g., variable domains or regions on the light and heavy chains (V_{L} and V_{H}, respectively) and constant domains or regions on the light and heavy chains (C_{L} and C_{H}, respectively). The N terminus of each chain defines a variable region of about 100 to 110 or more amino acids, referred to as the paratope, primarily responsible for antigen recognition, i.e., the antigen binding domain. Light chains are classified as either kappa or lambda. Heavy chains are classified as gamma, mu, alpha, delta, or epsilon, which in turn define the immunoglobulin classes, IgG, IgM, IgA, IgD and IgE, respectively. IgG antibodies are large molecules of about 150 kDa composed of four peptide chains. IgG antibodies contain two identical class 7 heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding domain. There are four IgG subclasses (IgG1, IgG2, IgG3, and IgG4) in humans, named in order of their abundance in serum (i.e., IgG1 is the most abundant). Typically, the antigen binding domain of an antibody will be most critical in specificity and affinity of binding to cancer cells.

Antibodies can exist as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region (see, e.g., Fundamental Immunology (Paul, editor, 7th edition, 2012)). While various antibody fragments are defined in terms of the digestion of an intact antibody, such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv), or those identified using phage display libraries (see, e.g., McCafferty et al., Nature, 348: 552-554 (1990)).

The term “antibody” specifically encompasses monoclonal antibodies (including full length monoclonal antibodies), polyclonal antibodies, multispecific antibodies (e.g., bispecific antibodies), and antibody fragments that exhibit the desired biological activity. An antibody that targets a particular antigen includes a bispecific or multispecific antibody with at least one antigen binding region that targets the particular antigen.

As used herein, the term “epitope” means any antigenic determinant or epitopic determinant of an antigen to which an antigen binding domain binds (i.e., at the paratope of the antigen binding domain). Antigenic determinants usually consist of chemically active surface groupings of molecules, such as amino acids or sugar side chains, and usually have specific three dimensional structural characteristics, as well as specific charge characteristics.

As used herein, “HER2” refers to the protein human epidermal growth factor receptor 2 (SEQ ID NO: 19), or an antigen with least about 70%, about 75%, about 80%, about 85%, about 90%, about 91%, about 92%, about 93%, about 94%, about 95%, about 96%, about 97%, about 98%, about 99%, or more sequence identity to SEQ ID NO: 19.

Percent (%) identity of sequences can be calculated, for example, as 100×[(identical positions)/min(TG_{A}, TG_{B})], where TG_{A} and TG_{B} are the sum of the number of residues and internal gap positions in peptide sequences A and B in the alignment that minimizes TG_{A} and TG_{B}. See, e.g., Russell et al., J. Mol Biol., 244: 332-350 (1994).

As used herein, the terms “Toll-like receptor” and “TLR” refer to any member of a family of highly-conserved mammalian proteins which recognizes pathogen-associated molecular patterns and acts as key signaling elements in innate immunity. TLR polypeptides share a characteristic structure that includes an extracellular domain that has leucine-rich repeats, a transmembrane domain, and an intracellular domain that is involved in TLR signaling.

The terms “Toll-like receptor 7” and “TLR7” refer to nucleic acids or polypeptides sharing at least about 70%, about 80%, about 90%, about 95%, about 96%, about 97%, about 98%, about 99%, or more sequence identity to a publicly-available TLR7 sequence, e.g., GenBank accession number AAZ99026 for human TLR7 polypeptide, or GenBank accession number AAK62676 for murine TLR7 polypeptide.

The terms “Toll-like receptor 8” and “TLR8” refer to nucleic acids or polypeptides sharing at least about 70%, about 80%, about 90%, about 95%, about 96%, about 97%, about 98%, about 99%, or more sequence identity to a publicly-available TLR7 sequence, e.g., GenBank accession number AAZ95441 for human TLR8 polypeptide, or GenBank accession number AAK62677 for murine TLR8 polypeptide.

A “TLR agonist” is a substance that binds, directly or indirectly, to a TLR (e.g., TLR7 and/or TLR8) to induce TLR signaling. Any detectable difference in TLR signaling can indicate that an agonist stimulates or activates a TLR. Signaling differences can be manifested, for example, as changes in the expression of target genes, in the phosphorylation of signal transduction components, in the intracellular localization of downstream elements such as nuclear factor-κB (NF-κB), in the association of certain components (such as IL-1 receptor associated kinase (IRAK)) with other proteins or intracellular structures, or in the biochemical activity of components such as kinases (such as mitogen-activated protein kinase (MAPK)).

As used herein, “Ab” of the immunoconjugates refers to an antibody construct that has an antigen binding domain that binds HER2 (e.g., trastuzumab (also known as HERCEPTIN™), a biosimilar thereof, or a biobetter thereof.

As used herein, the term “biosimilar” refers to an antibody construct that has active properties similar to the antibody construct previously approved (e.g., trastuzumab).

As used herein, the term “biobetter” refers to an approved antibody construct that is an improvement of a previously approved antibody construct (e.g., trastuzumab). The biobetter can have one or more modifications (e.g., an altered glycan profile, or a unique epitope) over the previously approved antibody construct.

As used herein, the term “amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid). The amino acids can be glycosylated (e.g., N-linked glycans, O-linked glycans, phosphoglycans, C-linked glycans, or glypiation) or deglycosylated.

Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, or an amino group) but have modified side-chain groups or modified peptide backbones, e.g., homoserine, norleucine, methionine sulfoxide, and methionine methyl sulfonium. “Amino acid mimetics” refer to chemical compounds that have a structure that is different from the general chemical structure of an amino acid, but that functions in a manner similar to a naturally-occurring amino acid.

Amino acids may be referred to herein by either the commonly known three letter symbols or by the one-letter symbols recommended by the IUPAC-IUB Biochemical Nomenclature Commission.

As used herein, the terms “treat,” “treatment,” and “treating” refer to any indicia of success in the treatment or amelioration of an injury, pathology, condition (e.g., cancer), or symptom (e.g., cognitive impairment), including any objective or subjective parameter such as abatement; remission; diminishing of symptoms or making the symptom, injury, pathology, or condition more tolerable to the subject; reduction in the rate of symptom progression; decreasing the frequency or duration of the symptom or condition; or, in some situations, preventing the onset of the symptom. The treatment or amelioration of symptoms can be based on any objective or subjective parameter, including, for example, the result of a physical examination. The treatment or amelioration of symptoms could be considered the standard of care at the time of treatment and/or consistent with the current practices in neoadjuvant, adjuvant, 1^{st}-line (1L), 2^{nd}-line (2L), 3^{rd}-line (3L), 4^{th}-line (4L), 5^{th}-line (5L), 6^{th}-line (6L), 7^{th}-line (7L), and beyond treatments for the cancer being treated. The treatment or amelioration of symptoms may be used with any type of tumor at any stage of disease.

The terms “cancer,” “neoplasm,” and “tumor” are used herein to refer to cells which exhibit autonomous, unregulated growth, such that the cells exhibit an aberrant growth phenotype characterized by a significant loss of control over cell proliferation. Cells of interest for detection, analysis, and/or treatment in the context of the invention include cancer cells (e.g., cancer cells from an individual with cancer), malignant cancer cells, pre-metastatic cancer cells, metastatic cancer cells, and non-metastatic cancer cells. Cancers of virtually every tissue are known. The phrase “cancer burden” refers to the quantum of cancer cells or cancer volume in a subject. Reducing cancer burden accordingly refers to reducing the number of cancer cells or the cancer cell volume in a subject. The term “cancer cell” as used herein refers to any cell that is a cancer cell (e.g., from any of the cancers for which an individual can be treated, e.g., isolated from an individual having cancer) or is derived from a cancer cell, e.g., clone of a cancer cell. For example, a cancer cell can be from an established cancer cell line, can be a primary cell isolated from an individual with cancer, can be a progeny cell from a primary cell isolated from an individual with cancer, and the like. In some embodiments, the term can also refer to a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, and plasmacytomas, and circulating cancers such as leukemias.

As used herein, the term “cancer” includes any form of cancer, including but not limited to, solid tumor cancers (e.g., lung, prostate, breast, gastric, bladder, colon, ovarian, pancreas, kidney, liver, glioblastoma, medulloblastoma, leiomyosarcoma, head & neck squamous cell carcinomas, melanomas, endometrial, salivary gland, and neuroendocrine) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors.

Carcinomas are malignancies that originate in the epithelial tissues. Epithelial cells cover the external surface of the body, line the internal cavities, and form the lining of glandular tissues. Examples of carcinomas include, but are not limited to, adenocarcinoma (cancer that begins in glandular (secretory) cells such as cancers of the breast, pancreas, lung, prostate, stomach, gastroesophageal junction, salivary gland, and colon), adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, salivary gland, and skin and other organs.

Soft tissue tumors are a highly diverse group of rare tumors that are derived from connective tissue. Examples of soft tissue tumors include, but are not limited to, alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; pleomorphic adenoma of soft tissue; and neoplasias derived from fibroblasts, myofibroblasts, histiocytes, vascular cells/endothelial cells, and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to, Askin's tumor; sarcoma botryoides; chondrosarcoma; Ewing's sarcoma; malignant hemangioendothelioma; malignant schwannoma; osteosarcoma; and soft tissue sarcomas (e.g., alveolar soft part sarcoma; angiosarcoma; cystosarcoma phyllodes, dermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); Kaposi's sarcoma; leiomyosarcoma; liposarcoma; lymphangiosarcoma; malignant peripheral nerve sheath tumor (MPNST); neurofibrosarcoma; synovial sarcoma; and undifferentiated pleomorphic sarcoma).

A teratoma is a type of germ cell tumor that may contain several different types of tissue (e.g., can include tissues derived from any and/or all of the three germ layers: endoderm, mesoderm, and ectoderm), including, for example, hair, muscle, and bone. Teratomas occur most often in the ovaries in women, the testicles in men, and the tailbone in children.

Melanoma is a form of cancer that begins in melanocytes (cells that make the pigment melanin). Melanoma may begin in a mole (skin melanoma), but can also begin in other pigmented tissues, such as in the eye or in the intestines.

Leukemias are cancers that start in blood-forming tissue, such as the bone marrow, and cause large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to, Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One category of lymphoma is Hodgkin lymphoma (HL), which is marked by the presence of a type of cell called the Reed-Sternberg cell. There are currently 6 recognized types of HL. Examples of Hodgkin lymphomas include nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to, AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to, gliomas (e.g., glioblastomas, astrocytomas, oligodendrogliomas, ependymomas, and the like), meningiomas, pituitary adenomas, and vestibular schwannomas, primitive neuroectodermal tumors (medulloblastomas).

The “pathology” of cancer includes all phenomena that compromise the well-being of the subject. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, and invasion of surrounding or distant tissues or organs, such as lymph nodes.

As used herein, the phrases “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs, therefore, tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part that is not directly connected to the organ of the original cancerous tumor. Metastasis can also be defined as several steps of a process, such as the departure of cancer cells from an original tumor site, and migration and/or invasion of cancer cells to other parts of the body.

As used herein the phrases “effective amount” and “therapeutically effective amount” refer to a dose of a substance such as an immunoconjugate that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition (McGraw-Hill, 2006); and Remington: The Science and Practice of Pharmacy, 22^{nd} Edition, (Pharmaceutical Press, London, 2012)).

As used herein, the terms “recipient,” “individual,” “subject,” “host,” and “patient” are used interchangeably and refer to any mammalian subject for whom diagnosis, treatment, or therapy is desired (e.g., humans). “Mammal” for purposes of treatment refers to any animal classified as a mammal, including humans, domestic and farm animals, and zoo, sports, or pet animals, such as dogs, horses, cats, cows, sheep, goats, pigs, camels, etc. In certain embodiments, the mammal is human.

The phrase “synergistic therapeutic agent” or “synergistic combination” in the context of this invention includes the combination of two immune modulators such as a receptor agonist, cytokine, and adjuvant polypeptide, that in combination elicit a synergistic effect on immunity relative to either administered alone. Particularly, the immunoconjugates disclosed herein comprise synergistic combinations of the therapeutic agent and antibody construct. These synergistic combinations upon administration elicit a greater effect on immunity, e.g., relative to when the antibody construct or therapeutic agent is administered in the absence of the other moiety. Further, a decreased amount of the immunoconjugate may be administered (as measured by the total number of antibody constructs or the total number of therapeutic agent administered as part of the immunoconjugate) compared to when either the antibody construct or therapeutic agent is administered alone.

As used herein, the term “administering” refers to parenteral, intravenous, intraperitoneal, intramuscular, intratumoral, intralesional, intranasal, or subcutaneous administration, oral administration, administration as a suppository, topical contact, intrathecal administration, or the implantation of a slow-release device, e.g., a mini-osmotic pump, to the subject.

The terms “about” and “around,” as used herein to modify a numerical value, indicate a close range surrounding the numerical value. Thus, if “X” is the value, “about X” or “around X” indicates a value of from 0.9X to 1.1X, e.g., from 0.95X to 1.05X or from 0.99X to 1.01X. A reference to “about X” or “around X” specifically indicates at least the values X, 0.95X, 0.96X, 0.97X, 0.98X, 0.99X, 1.01X, 1.02X, 1.03X, 1.04X, and 1.05X. Accordingly, “about X” and “around X” are intended to teach and provide written description support for a claim limitation of, e.g., “0.98X.”

As used herein, the abbreviation “AUC” refers to “area under the curve” and can be determined using biological samples analyzed with LC/MS/MS. Accordingly, the AUC can be determined by any suitable LC/MS/MS apparatus. The AUC can be calculated from a single exposure, multi-dose, and/or a steady state exposure curve. Alternatively, or in addition to, the AUC can be calculated from the average (mean), time-weighted average, and/or instantaneous drug exposure curve. Typically, the AUC refers to the average area under the curve for a single dose drug exposure over a period of 24 hours.

As used herein, the phrase “coefficient of variation” refers to the relative standard deviation and is calculated as follows:

$${C}_{V} = \frac{σ}{μ}$$

wherein C_{V} is the coefficient of variation, σ is the standard deviation, and p is the mean.

As used herein, the abbreviation “Cₘₐₓ” refers to the maximum plasma concentration.

As used herein, the abbreviation “t_{1/2}” refers to the biological half-life.

### Immunoconjugate Dosing Regimen

The methods can include treating cancer in a subject comprising administering from about 0.01 mg/kg to about 100 mg/kg of the immunoconjugate, or a pharmaceutically acceptable salt thereof, to the subject. In this regard, the methods can include administering the immunoconjugate, or pharmaceutically acceptable salt thereof, to provide a dose of from about 0.1 mg/kg to about 90 mg/kg, from about 0.1 mg/kg to about 80 mg/kg, from about 0.1 mg/kg to about 70 mg/kg, from about 0.1 mg/kg to about 60 mg/kg, from about 0.1 mg/kg to about 50 mg/kg, from about 0.1 mg/kg to about 40 mg/kg, from about 0.1 mg/kg to about 30 mg/kg, from about 0.1 mg/kg to about 25 mg/kg, from about 0.1 mg/kg to about 0.2 mg/kg, from about 0.25 mg/kg to 0.75 about mg/kg, from about 1 mg/kg to about 3 mg/kg, from about 4 mg/kg to about 6 mg/kg, from about 4.5 mg/kg to about 5.5 mg/kg, from about 8 mg/kg to about 12 mg/kg, from about 9 mg/kg to about 11 mg/kg, from about 10 mg/kg to about 14 mg/kg, from about 11 mg/kg to about 13 mg/kg, from about 17 mg/kg to about 23 mg/kg, from about 18 mg/kg to about 22 mg/kg, or from about 19 mg/kg to about 21 mg/kg. In some embodiments, the methods include administering about 0.15 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg of the immunoconjugate, or a pharmaceutically acceptable salt thereof, to the subject.

In some embodiments, the immunoconjugate, or a pharmaceutically acceptable salt thereof, is administered from about every 3 to about every 45 days (e.g., about every 3 days, about every 4 days, about every 5 days, about every 6 days, about every 7 days, about every 8 days, about every 9 days, about every 10 days, about every 11 days, about every 12 days, about every 13 days, about every 14 days, about every 15 days, about every 16 days, about every 17 days, about every 18 days, about every 19 days, about every 20 days, about every 21 days, about every 22 days, about every 23 days, about every 24 days, about every 25 days, about every 26 days, about every 27 days, about every 28 days, about every 29 days, about every 30 days, about every 31 days, about every 32 days, about every 33 days, about every 34 days, about every 35 days, about every 36 days, about every 37 days, about every 38 days, about every 39 days, about every 40 days, about every 41 days, about every 42 days, about every 43 days, about every 44 days, or about every 45 days). In some embodiments, the immunoconjugate, or a pharmaceutically acceptable salt thereof, is administered from about every 3 to about every 35 days. In some embodiments, the immunoconjugate, or a pharmaceutically acceptable salt thereof, is administered every 1, 2, 3, 4, 5, 6, or 7 weeks, or every month. In some embodiments, the immunoconjugate, or a pharmaceutically acceptable salt thereof, is administered from about every 5 to about every 9 days, from about every 6 to about every 8 days, from about every 13 to about every 15 days, from about every 12 to about every 16 days, from about every 20 to about every 22 days, from about every 19 to about every 23 days, from about every 27 to about every 29 days, from about every 26 to about every 30 days, or from about every 33 to about every 37 days. In some embodiments, the immunoconjugate, or a pharmaceutically acceptable salt thereof, is administered about every 7 days, about every about 14 days, about every 21 days, about every 28 days, about every 35 days, or about every 42 days.

The immunoconjugate, or a pharmaceutically acceptable salt thereof, can be administered to the subject as an initial loading dose followed by one or more maintenance doses. For example, the immunoconjugate, or a pharmaceutically acceptable salt thereof, can be administered as a loading dose to the subject at about 5 mg/kg, about 8 mg/kg, about 12 mg/kg, about 15 mg/kg, or about 20 mg/kg by IV infusion. The loading dose may be a higher or lower dose than the one or more maintenance doses. The loading dose may be administered to the patient using a similar or different suitable means than the one or more maintenance doses.

The immunoconjugate, or a pharmaceutically acceptable salt thereof, can be administered to the subject using any suitable means including parenteral, intravenous, intraperitoneal, intramuscular, intratumoral, intralesional, intranasal, or subcutaneous administration, oral administration, administration as a suppository, topical contact, intrathecal administration, or the implantation of a slow release device, e.g., a miniosmotic pump, to the subject.

In some embodiments, the immunoconjugate, or a pharmaceutically acceptable salt thereof, is administered subcutaneously.

In some embodiments, the immunoconjugate, or a pharmaceutically acceptable salt thereof, is administered intravenously (e.g., IV infusion). In some embodiments, the immunoconjugate, or a pharmaceutically acceptable salt thereof, is administered to the subject intravenously over about 1 to about 240 minutes. In this regard, the immunoconjugate, or a pharmaceutically acceptable salt thereof, can be administered over about 5 to about 55 minutes, over about 10 to about 50 minutes, over about 15 to about 45 minutes, over about 20 to about 40 minutes, over about 25 to about 35 minutes, over about 30 minutes to the subject, over about 30 to about 90 minutes, over about 35 to about 85 minutes, over about 40 to about 80 minutes, over about 45 to about 75 minutes, over about 50 to about 70 minutes, over about 55 to about 65 minutes, over about 60 minutes, over about 90 to about 150 minutes, over about 95 to about 145 minutes, over about 100 to about 140 minutes, over about 105 to about 135 minutes, over about 110 to about 130 minutes, over about 115 to about 125 minutes, over about 120 minutes, over about 150 to about 210 minutes, over about 155 to about 205 minutes, over about 160 to about 200 minutes, over about 165 to about 195 minutes, over about 170 to about 190 minutes, over about 175 to about 185 minutes, over about 180 minutes, over about 210 to about 270 minutes, over about 215 to about 265 minutes, over about 220 to about 260 minutes, over about 225 to about 255 minutes, over about 230 to about 250 minutes, over about 235 to about 245 minutes, or over about 240 minutes.

The immunoconjugate, or a pharmaceutically acceptable salt thereof, can be administered to the subject for any suitable length of time. For example, the immunoconjugate, or pharmaceutically acceptable salt thereof, can be administered to the subject one time or multiple times. If the immunoconjugate, or pharmaceutically acceptable salt thereof, is administered multiple times, the immunoconjugate, or a pharmaceutically acceptable salt thereof, can be administered for a duration of from about 1 month to about 48 months (e.g., from about 1 to about 45 months, from about 1 to about 40 months, from about 1 to about 35 months, from about 1 to about 30 months, from about 1 to about 25 months, from about 1 to about 20 months, from about 1 to about 15 months, from about 1 to about 12 months, from about 1 to about 10 months, from about 1 to about 5 months, from about 1 to about 4 months, from about 1 to about 3 months, from about 1 to about 2 months, or about 1 month).

The immunoconjugate, or a pharmaceutically acceptable salt thereof, can be administered to the subject at 0.15 mg/kg every week by IV infusion. The immunoconjugate, or a pharmaceutically acceptable salt thereof, can be administered to the subject at 0.5 mg/kg every week by IV infusion. The immunoconjugate, or a pharmaceutically acceptable salt thereof, can be administered to the subject at 2 mg/kg every week by IV infusion. The immunoconjugate, or a pharmaceutically acceptable salt thereof, can be administered to the subject at 5 mg/kg every week by IV infusion. The immunoconjugate, or a pharmaceutically acceptable salt thereof, can be administered to the subject at 8 mg/kg every week by IV infusion. The immunoconjugate, or a pharmaceutically acceptable salt thereof, can be administered to the subject at 12 mg/kg every week by IV infusion. The immunoconjugate, or a pharmaceutically acceptable salt thereof, can be administered to the subject at 20 mg/kg every week by IV infusion.

The immunoconjugate, or a pharmaceutically acceptable salt thereof, can be administered to the subject at 0.15 mg/kg every 2 weeks by IV infusion. The immunoconjugate, or a pharmaceutically acceptable salt thereof, can be administered to the subject at 0.5 mg/kg every 2 weeks by IV infusion. The immunoconjugate, or a pharmaceutically acceptable salt thereof, can be administered to the subject at 2 mg/kg every 2 weeks by IV infusion. The immunoconjugate, or a pharmaceutically acceptable salt thereof, can be administered to the subject at 5 mg/kg every 2 weeks by IV infusion. The immunoconjugate, or a pharmaceutically acceptable salt thereof, can be administered to the subject at 8 mg/kg every 2 weeks by IV infusion. The immunoconjugate, or a pharmaceutically acceptable salt thereof, can be administered to the subject at 12 mg/kg every 2 weeks by IV infusion. The immunoconjugate, or a pharmaceutically acceptable salt thereof, can be administered to the subject at 20 mg/kg every 2 weeks by IV infusion.

The immunoconjugate, or a pharmaceutically acceptable salt thereof, can be administered to the subject at 0.15 mg/kg every 3 weeks by IV infusion. The immunoconjugate, or a pharmaceutically acceptable salt thereof, can be administered to the subject at 0.5 mg/kg every 3 weeks by IV infusion. The immunoconjugate, or a pharmaceutically acceptable salt thereof, can be administered to the subject at 2 mg/kg every 3 weeks by IV infusion. The immunoconjugate, or a pharmaceutically acceptable salt thereof, can be administered to the subject at 5 mg/kg every 3 weeks by IV infusion. The immunoconjugate, or a pharmaceutically acceptable salt thereof, can be administered to the subject at 8 mg/kg every 3 weeks by IV infusion. The immunoconjugate, or a pharmaceutically acceptable salt thereof, can be administered to the subject at 12 mg/kg every 3 weeks by IV infusion. The immunoconjugate, or a pharmaceutically acceptable salt thereof, can be administered to the subject at 20 mg/kg every 3 weeks by IV infusion.

### Immunoconjugates

The invention provides an immunoconjugate of formula: Ab-[TA]ᵣ or a pharmaceutically acceptable salt thereof, wherein “Ab” is an antibody construct that has an antigen binding domain that binds human epidermal growth factor receptor type 2 (HER2) and “TA” is a therapeutic agent of formula:

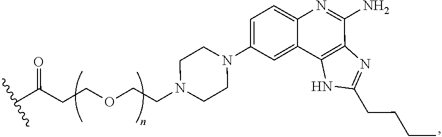

wherein n is from about 2 to about 25 (e.g., about 2 to about 16, about 6 to about 25, about 6 to about 16, about 8 to about 25, about 8 to about 16, about 6 to about 12, about 8 to about 12, or about 10), and r is an average therapeutic agent to antibody ratio from 1 to 10. “Ab” can be any suitable antibody construct that has an antigen binding domain that binds HER2, such as, for example, trastuzumab and pertuzumab. In certain embodiments, “Ab” is trastuzumab (also known as HERCEPTIN™), a biosimilar thereof, or a biobetter thereof. For example, “Ab” can be MYL-14010, ABP 980, BCD-022, CT-P6, EG12014, HD201, ONS-1050, PF-05280014, ONTRUZANT™ (SB3), Saiputing, HERZUMA™ (CT-P6), or HLX02. In preferred embodiments, “Ab” is trastuzumab (also known as HERCEPTIN™).

Generally, the immunoconjugates of the invention have an average therapeutic agent to antibody ratio of from 1 to 10. The average therapeutic agent to antibody is designated with subscript “r.” Generally, each of the therapeutic agents is conjugated to the antibody construct at an amine of a lysine residue of the antibody construct. However, it will be understood to a skilled artisan that there can be occasional off target conjugations such that a therapeutic agent can be bound to the antibody construct at an amino acid other than lysine. In an embodiment, r is 1, such that there is only one therapeutic agent bound to the antibody construct (i.e., a homogenous conjugation of one). In some embodiments, r is any number from about 1 to about 10 (e.g., about 2 to about 10, about 2 to about 9, about 3 to about 9, about 4 to about 9, about 5 to about 9, about 6 to about 9, about 3 to about 8, about 3 to about 7, about 3 to about 6, about 4 to about 8, about 4 to about 7, about 4 to about 6, about 5 to about 6, about 1 to about 6, about 1 to about 4, about 2 to about 4, or about 1 to about 3). In preferred embodiments, the immunoconjugates have an average therapeutic agent to antibody construct ratio (i.e., subscript “r” can be) from about 1 to about 4 or about 2 to about 3. The desirable average therapeutic agent to antibody construct ratio (i.e., the value of the subscript “r”) can be determined by a skilled artisan depending on the desired effect of the treatment.

Generally, the immunoconjugates of the invention comprise about 2 to about 25 (e.g., about 2 to about 16, about 6 to about 25, about 6 to about 16, about 8 to about 25, about 8 to about 16, about 6 to about 12, or about 8 to about 12) ethylene glycol units in the therapeutic agent, as designated with subscript “n.” Accordingly, the immunoconjugates of the invention can comprise at least 2 ethylene glycol groups (e.g., at least 3 ethylene glycol groups, at least 4 ethylene glycol groups, at least 5 ethylene glycol groups, at least 6 ethylene glycol groups, at least 7 ethylene glycol groups, at least 8 ethylene glycol groups, at least 9 ethylene glycol groups, or at least 10 ethylene glycol groups). Accordingly, the immunoconjugate can comprise from about 2 to about 25 ethylene glycol units in the therapeutic agent, for example, from about 6 to about 25 ethylene glycol units, from about 6 to about 16 ethylene glycol units, from about 8 to about 25 ethylene glycol units, from about 8 to about 16 ethylene glycol units, from about 8 to about 12 ethylene glycol units, or from about 8 to about 12 ethylene glycol units. In certain embodiments, the immunoconjugate comprises a di(ethylene glycol) group, a tri(ethylene glycol) group, a tetra(ethylene glycol) group, 5 ethylene glycol groups, 6 ethylene glycol groups, 7 ethylene glycol groups, 8 ethylene glycol groups, 9 ethylene glycol groups, 10 ethylene glycol groups, 11 ethylene glycol groups, 12 ethylene glycol groups, 13 ethylene glycol groups, 14 ethylene glycol groups, 15 ethylene glycol groups, 16 ethylene glycol groups, 24 ethylene glycol groups, or 25 ethylene glycol groups. In preferred embodiments, the immunoconjugate comprises 6 ethylene glycol groups, 8 ethylene glycol groups, 10 ethylene glycol groups, or 12 ethylene glycol groups (i.e., about 6 ethylene glycol groups to about 12 ethylene glycol groups) in the therapeutic agent.

The therapeutic agent can be conjugated to the antibody construct that has an antigen binding domain that binds HER2 (e.g., trastuzumab, pertuzumab, biosimilars thereof, and biobetters thereof) via an amine of a lysine residue of the antibody construct. Accordingly, the immunoconjugates of the invention can be represented by the following formula:

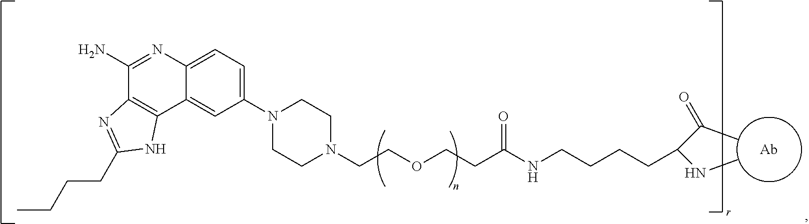

wherein n is from about 2 to about 25 and

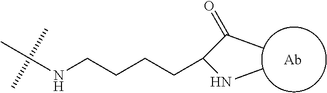

is an antibody construct that has an antigen binding domain that binds HER2 with residue

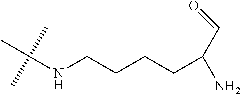

representing a lysine residue of the antibody construct, wherein “ [symbol] ” represents a point of attachment to the therapeutic agent.

The therapeutic agent can be bound to any suitable residue of the antibody construct, but desirably is bound to any lysine residue of the antibody construct. For example, the therapeutic agent can be bound to one or more of K103, K107, K149, K169, K183, and/or K188 of the light chain of the antibody construct, as numbered using the Kabat numbering system. Alternatively, or additionally, the therapeutic agent can be bound to one or more of K30, K43, K65, K76, K136, K216, K217, K225, K293, K320, K323, K337, K395, and/or K417 of the heavy chain of the antibody construct, as numbered using the Kabat numbering system. Generally, the therapeutic agent is predominantly bound at K107 or K188 of the light chain of the antibody construct, or K30, K43, K65, or K417 of the heavy chain of the antibody construct. In certain embodiments, the therapeutic agent is bound at K188 of the light chain of the antibody construct, and optionally one or more other lysine residues of the antibody construct.

An immunoconjugate, or a pharmaceutically acceptable salt thereof, as described herein can provide an unexpectedly increased activation response of an antigen presenting cell (APC). This increased activation can be detected in vitro or in vivo. In some embodiments, the increased APC activation can be detected in the form of a reduced time to achieve a specified level of APC activation. For example, in an in vitro assay, % APC activation can be achieved at an equivalent dose with an immunoconjugate within about 1%, about 10%, about 20%, about 30%, about 40%, or about 50% of the time required to obtain the same or similar percentage of APC activation with a mixture of unconjugated antibody construct and therapeutic agent, under otherwise identical concentrations and conditions. In some embodiments, an immunoconjugate can activate APCs (e.g., dendritic cells) and/or NK cells in a reduced amount of time. For example, in some embodiments, a mixture of unconjugated antibody construct and therapeutic agent can activate APCs (e.g., dendritic cells) and/or NK cells and/or induce dendritic cell differentiation after incubation with the mixture for 2, 3, 4, 5, 1-5, 2-5, 3-5, or 4-7 days, while, in contrast, immunoconjugates described herein can activate and/or induce differentiation within 4 hours, 8 hours, 12 hours, 16 hours, or 1 day, under otherwise identical concentrations and conditions. Alternatively, the increased APC activation can be detected in the form of a reduced concentration of immunoconjugate required to achieve an amount (e.g., percent APCs), level (e.g., as measured by a level of upregulation of a suitable marker) or rate (e.g., as detected by a time of incubation required to activate) of APC activation.

In some embodiments, the immunoconjugates of the invention provide more than an about 5% increase in activity compared to a mixture of unconjugated antibody construct and therapeutic agent, under otherwise identical conditions. In other embodiments, the immunoconjugates of the invention provide more than an about 10%, about 15%, about 20%, about 25%, about 30%, about 35%, about 40%, about 45%, about 50%, about 55%, about 60%, about 65%, or about 70% increase in activity compared to a mixture of unconjugated antibody construct and therapeutic agent, under otherwise identical conditions. The increase in activity can be assessed by any suitable means, many of which are known to those ordinarily skilled in the art and can include myeloid activation, assessment by cytokine secretion, or a combination thereof.

In some embodiments, the invention provides an immunoconjugate of formula: Ab-[TA]ᵣ or a pharmaceutically acceptable salt thereof, wherein “Ab” is an antibody construct that has an antigen binding domain that binds human epidermal growth factor receptor type 2 (HER2) and “TA” is a therapeutic agent of formula:

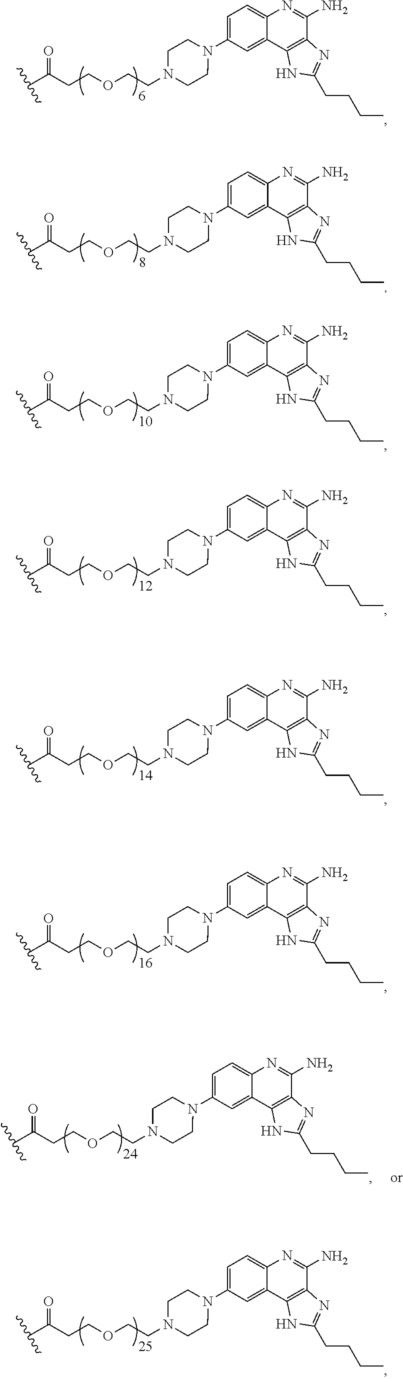

wherein subscript r is an average therapeutic agent to antibody ratio from about 1 to about 10.

In certain embodiments, the invention provides an immunoconjugate of formula: Ab-[TA]ᵣ or a pharmaceutically acceptable salt thereof, wherein “Ab” is trastuzumab (also known as HERCEPTIN™), pertuzumab, a biosimilar thereof, or a biobetter thereof (for example, “Ab” can be MYL-14010, ABP 980, BCD-022, CT-P6, EG12014, HD201, ONS-1050, PF-05280014, ONTRUZANT™ (SB3), Saiputing, HERZUMA™ (CT-P6), or HLX02) and “TA” is a therapeutic agent of formula:

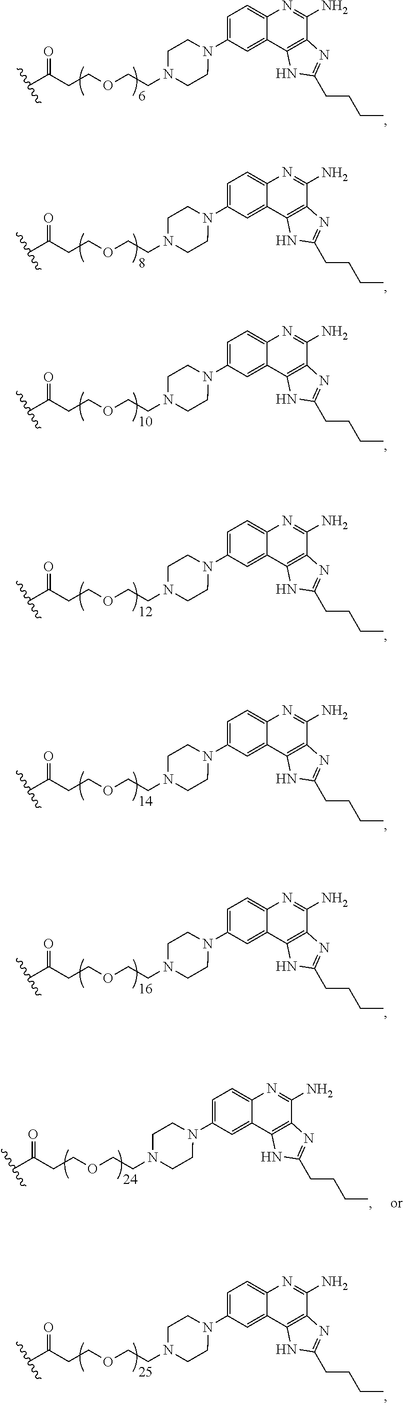

wherein subscript r is an average therapeutic agent to antibody ratio from about 1 to about 10.

In preferred embodiments, the invention provides an immunoconjugate of formula: Ab-[TA]ᵣ or a pharmaceutically acceptable salt thereof, wherein “Ab” is trastuzumab (also known as HERCEPTIN™) and “TA” is a therapeutic agent of formula:

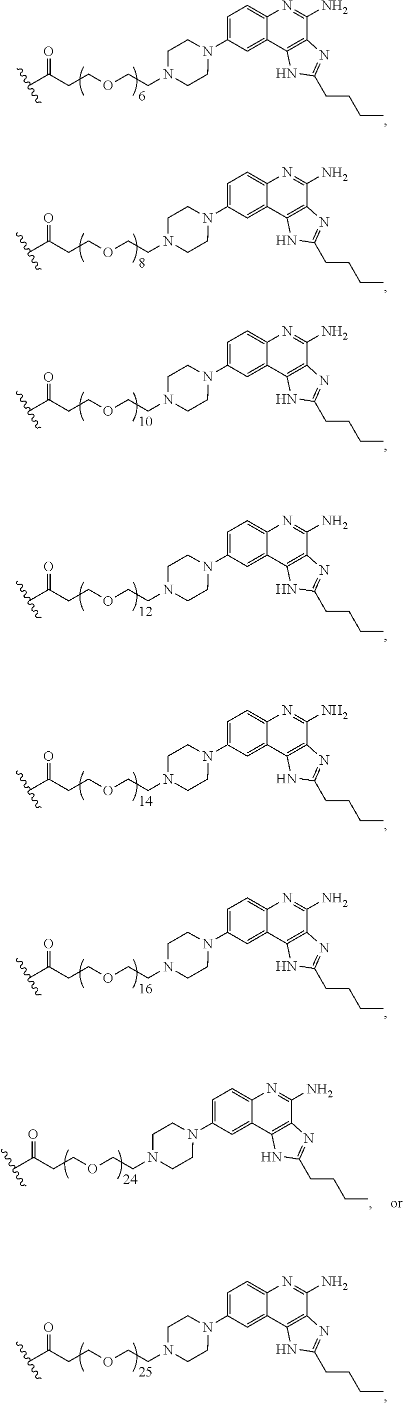

wherein subscript r is an average therapeutic agent to antibody ratio from about 1 to about 10.

In a preferred embodiment, the invention provides an immunoconjugate of formula: Ab-[TA]ᵣ or a pharmaceutically acceptable salt thereof, wherein “Ab” is trastuzumab (also known as HERCEPTIN™) and “TA” is a therapeutic agent of formula:

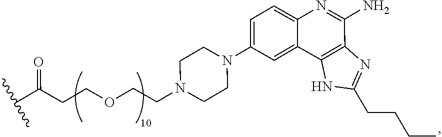

wherein subscript r is an average therapeutic agent to antibody ratio from about 1 to about 10. This immunoconjugate is referred to herein as BDC-1001.

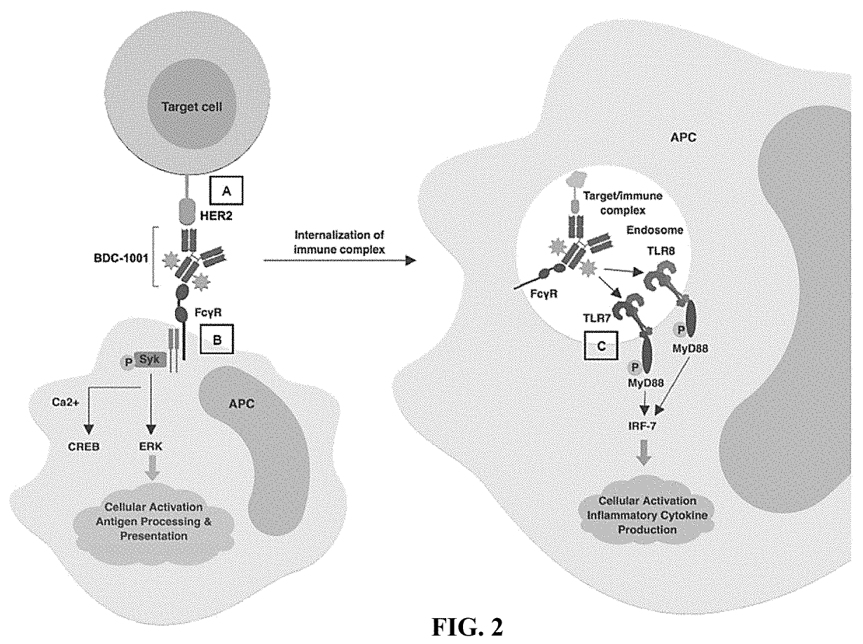

Without being bound to any particular theory, it is believed that an immunoconjugate, such as BDC-1001 binds to HER2 expressing tumor cells via the “Ab” of BDC-1001 leading to tumor cell killing and phagocytosis. The therapeutic agent of BDC-1001 activates myeloid APCs such as macrophages and dendritic cells which leads to increased cytotoxicity, processing, and presentation of tumor neoantigens that subsequently stimulate T cell-mediated immunity (see FIG. 2).

In some embodiments, administration of the immunoconjugate, or a pharmaceutically acceptable salt thereof, results in increased plasma levels of cytokines and/or chemokines, such as those consistent with TLR7/8 and myeloid cell activation. In some embodiments, administration of the immunoconjugate, or a pharmaceutically acceptable salt thereof, results in increased plasma levels of monocyte chemoattractant protein-1 (MCP-1) in the subject. In some embodiments, administration of the immunoconjugate, or a pharmaceutically acceptable salt thereof, results in increased plasma levels of macrophage inflammatory protein 1α (MIP1α) in the subject. In some embodiments, administration of the immunoconjugate, or a pharmaceutically acceptable salt thereof, results in increased plasma levels of interferon gamma-induced protein 10 (IP-10) in the subject.

In some embodiments, administration of the immunoconjugate, or a pharmaceutically acceptable salt thereof, results in increased plasma levels of indicators of TLR activation. In some embodiments, administration of the immunoconjugate, or a pharmaceutically acceptable salt thereof, results in increased plasma levels of TNFα.

### Therapeutic Agents

The immunoconjugate of the invention comprises a therapeutic agent of formula:

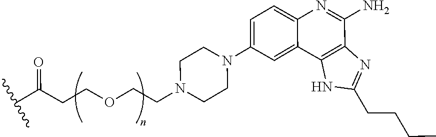

wherein n is from about 2 to about 25 and “ [symbol] ” represents a point of attachment of the therapeutic agent to the antibody construct.

The therapeutic agent described herein is an adjuvant, more specifically, is a TLR agonist. In some embodiments, the cancer treated by the methods of the invention are susceptible to a pro-inflammatory response induced by TLR7 and/or TLR8 agonism.

### Antigen Binding Domain and Fc Domain

The immunoconjugates of the invention comprise an antibody construct that comprises an antigen binding domain that binds HER2. In some embodiments, the antibody construct further comprises an Fc domain. In certain embodiments, the antibody construct is an antibody. In certain embodiments, the antibody construct is a fusion protein.

The antigen binding domain can be a single-chain variable region fragment (scFv). A single-chain variable region fragment (scFv), which is a truncated Fab fragment including the variable (V) domain of an antibody heavy chain linked to a V domain of a light antibody chain via a synthetic peptide, can be generated using routine recombinant DNA technology techniques. Similarly, disulfide-stabilized variable region fragments (dsFv) can be prepared by recombinant DNA technology.

An embodiment of the invention provides antibody construct or antigen binding domain which specifically recognizes and binds to HER2 (SEQ ID NO: 19). The antibody construct or antigen binding domain may comprise one or more variable regions (e.g., two variable regions) of an antigen binding domain of an anti-HER2 antibody, each variable region comprising a CDR1, a CDR2, and a CDR3.

An embodiment of the invention provides an antibody construct or antigen binding domain comprising the CDR regions of trastuzumab. In this regard, the antigen binding domain may comprise a first variable region comprising a CDR1 comprising the amino acid sequence of SEQ ID NO: 20 (CDR1 of first variable region), a CDR2 comprising the amino acid sequence of SEQ ID NO: 21 (CDR2 of first variable region), and a CDR3 comprising the amino acid sequence of SEQ ID NO: 22 (CDR3 of first variable region), and a second variable region comprising a CDR1 comprising the amino acid sequence of SEQ ID NO: 23 (CDR1 of second variable region), a CDR2 comprising the amino acid sequence of SEQ ID NO: 24 (CDR2 of second variable region), and a CDR3 comprising the amino acid sequence of SEQ ID NO: 25 (CDR3 of second variable region). In this regard, the antibody construct can comprise (i) all of SEQ ID NOs: 20-23, (ii) all of SEQ ID NOs: 23-25, or (iii) all of SEQ ID NOs: 20-25. Preferably, the antigen binding domain comprises all of SEQ ID NOs: 20-25.

In an embodiment of the invention, the antigen binding domain comprising the CDR regions of trastuzumab further comprises the framework regions of the trastuzumab. In this regard, the antigen binding domain comprising the CDR regions of trastuzumab further comprises the amino acid sequence of SEQ ID NO: 26 (framework region (FR) 1 of first variable region), the amino acid sequence of SEQ ID NO: 27 (FR2 of first variable region), the amino acid sequence of SEQ ID NO: 28 (FR3 of first variable region), the amino acid sequence of SEQ ID NO: 29 (FR4 of first variable region), the amino acid sequence of SEQ ID NO: 30 (FR1 of second variable region), the amino acid sequence of SEQ ID NO: 31 (FR2 of second variable region), the amino acid sequence of SEQ ID NO: 32 (FR3 of second variable region), and the amino acid sequence of SEQ ID NO: 33 (FR4 of second variable region). In this regard, the antibody construct or antigen binding domain can comprise (i) all of SEQ ID NOs: 20-22 and 26-29, (ii) all of SEQ ID NOs: 23-25 and 30-33; or (iii) all of SEQ ID NOs: 20-25 and 26-33.

In an embodiment of the invention, the antigen binding domain comprises one or both variable regions of trastuzumab. In this regard, the first variable region may comprise SEQ ID NO: 48. The second variable region may comprise SEQ ID NO: 49. Accordingly, in an embodiment of the invention, the antigen binding domain comprises SEQ ID NO: 48, SEQ ID NO: 49, or both SEQ ID NOs: 48 and 49. Preferably, the antigen binding domain comprises both of SEQ ID NOs: 48-49.

In an embodiment of the invention, the antigen binding domain comprises the CDR regions of pertuzumab. In this regard, the antigen binding domain may comprise a first variable region comprising a CDR1 comprising the amino acid sequence of SEQ ID NO: 34 (CDR1 of first variable region), a CDR2 comprising the amino acid sequence of SEQ ID NO: 35 (CDR2 of first variable region), and a CDR3 comprising the amino acid sequence of SEQ ID NO: 36 (CDR3 of first variable region), and a second variable region comprising a CDR1 comprising the amino acid sequence of SEQ ID NO: 37 (CDR1 of second variable region), a CDR2 comprising the amino acid sequence of SEQ ID NO: 38 (CDR2 of second variable region), and a CDR3 comprising the amino acid sequence of SEQ ID NO: 39 (CDR3 of second variable region). In this regard, the antigen binding domain can comprise (i) all of SEQ ID NOs: 34-36, (ii) all of SEQ ID NOs: 37-39, or (iii) all of SEQ ID NOs: 34-39. Preferably, the antigen binding domain comprises all of SEQ ID NOs: 34-39.

In an embodiment of the invention, the antigen binding domain comprising the CDR regions of pertuzumab further comprises the framework regions of the pertuzumab. In this regard, the antigen binding domain comprising the CDR regions of the pertuzumab further comprises the amino acid sequence of SEQ ID NO: 40 (FR1 of first variable region), the amino acid sequence of SEQ ID NO: 41 (FR2 of first variable region), the amino acid sequence of SEQ ID NO: 42 (FR3 of first variable region), the amino acid sequence of SEQ ID NO: 43 (FR4 of first variable region), the amino acid sequence of SEQ ID NO: 44 (FR1 of second variable region), the amino acid sequence of SEQ ID NO: 45 (FR2 of second variable region), the amino acid sequence of SEQ ID NO: 46 (FR3 of second variable region), and the amino acid sequence of SEQ ID NO: 47 (FR4 of second variable region). In this regard, the antigen binding domain can comprise (i) all of SEQ ID NOs: 34-36 and 40-43, (ii) all of SEQ ID NOs: 37-39 and 44-47; or (iii) all of SEQ ID NOs: 34-39 and 40-47.

In an embodiment of the invention, the antigen binding domain comprises one or both variable regions of pertuzumab. In this regard, the first variable region may comprise SEQ ID NO: 50. The second variable region may comprise SEQ ID NO: 51. Accordingly, in an embodiment of the invention, the antigen binding domain comprises SEQ ID NO: 50, SEQ ID NO: 51, or both SEQ ID NOs: 50 and 51. Preferably, the antigen binding domain comprises both of SEQ ID NOs: 50-51.

The scope of the embodiments of the invention includes functional variants of the antibody construct and antigen binding domain described herein. The term “functional variant” as used herein refers to an antibody construct having an antigen binding domain with substantial or significant sequence identity or similarity to a parent antibody construct or antigen binding domain, which functional variant retains the biological activity of the parent antibody construct or antigen binding domain, respectively, of which it is a variant. Functional variants encompass, for example, those variants of the antibody construct or antigen binding domain described herein (the parent antibody construct or antigen binding domain) that retain the ability to recognize target cells expressing HER2 to a similar extent, the same extent, or to a higher extent, as the parent antibody construct or antigen binding domain.

In reference to the antibody construct or antigen binding domain, the functional variant can, for instance, be at least about 30%, about 50%, about 75%, about 80%, about 85%, about 90%, about 91%, about 92%, about 93%, about 94%, about 95%, about 96%, about 97%, about 98%, about 99% or more identical in amino acid sequence to the parent antibody construct or antigen binding domain, respectively.

A functional variant can, for example, comprise the amino acid sequence of the parent antibody construct or antigen binding domain with at least one conservative amino acid substitution. Alternatively, or additionally, the functional variant can comprise the amino acid sequence of the parent antibody construct or antigen binding domain with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent antibody construct or antigen binding domain, respectively.

Amino acid substitutions of the inventive antibody constructs or antigen binding domains are preferably conservative amino acid substitutions. Conservative amino acid substitutions are known in the art and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g., Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The antibody construct or antigen binding domain can consist essentially of the specified amino acid sequence or sequences described herein, such that other components, e.g., other amino acids, do not materially change the biological activity of the antibody construct or antigen binding domain functional variant.

The antibody constructs and antigen binding domains of embodiments of the invention (including functional portions and functional variants) can be of any length, i.e., can comprise any number of amino acids, provided that the antibody constructs (or functional portions or functional variants thereof) retain their biological activity, e.g., the ability to specifically bind to HER2, detect cancer cells in a mammal, or treat or prevent cancer in a mammal, etc. For example, the antibody construct or antigen binding domain can be about 50 to about 5,000 amino acids long, such as 50, 70, 75, 100, 125, 150, 175, 200, 300, 400, 500, 600, 700, 800, 900, 1,000, or more amino acids in length.

The antibody constructs and antigen binding domains of embodiments of the invention (including functional portions and functional variants thereof) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The antibody constructs of embodiments of the invention (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized.

In some embodiments, the antibody construct is a monoclonal antibody of a defined sub-class (e.g., IgG₁, IgG₂, IgG₃, IgG₄, IgA₁, or IgA₂). Typically, the antibody construct is an IgG₁ antibody. Various combinations of different subclasses, in different relative proportions, can be obtained by those of skill in the art. In some embodiments, a specific subclass or a specific combination of different subclasses can be particularly effective at cancer treatment or tumor size reduction. Accordingly, some embodiments of the invention provide immunoconjugates wherein the antibody is a monoclonal antibody. In some embodiments, the monoclonal antibody is a humanized monoclonal antibody.

In some embodiments, the antibody construct or antigen binding domain binds to HER2 on a cancer or immune cell at a higher affinity than a corresponding HER2 antigen on a non-cancer cell. For example, the antibody construct or antigen binding domain may preferentially recognize HER2 containing a polymorphism that is found on a cancer or immune cell as compared to recognition of a corresponding wild-type HER2 antigen on the non-cancer cell. In some embodiments, the antibody construct or antigen binding domain binds a cancer cell with greater avidity than a non-cancer cell. For example, the cancer cell can express a higher density of HER2, thereby providing for a higher affinity binding of a multivalent antibody to the cancer cell.

In some embodiments, the antibody construct or antigen binding domain does not significantly bind non-cancer antigens (e.g., the antibody binds one or more non-cancer antigens with at least 10, 100, 1,000, 10,000, 100,000, or 1,000,000-fold lower affinity (higher Kd) than HER2). In some embodiments, the corresponding non-cancer cell is a cell of the same tissue or origin that is not hyperproliferative or otherwise cancerous. HER2 need not be specific to the cancer cell or even enriched in cancer cells relative to other cells (e.g., HER2 can be expressed by other cells). Thus, in the phrase “an antibody construct that specifically binds to an antigen of a cancer cell,” the term “specifically” refers to the specificity of the antibody construct and not to the uniqueness of the presence of HER2 in that particular cell type.

Any HER2 expressing cancer is a suitable cancer to be treated by the subject methods and compositions. As used herein “HER2 expression” refers to a cell that has a HER2 receptor on the cell's surface. For example, a cell may have from about 20,000 to about 50,000 HER2 receptors on the cell's surface. As used herein “HER2 overexpression” refers to a cell that has more than about 50,000 HER2 receptors (IHC1+). For example, a cell with 2, 5, 10, 100, 1,000, 10,000, 100,000, or 1,000,000 times the number of HER2 receptors as compared to corresponding non-cancer cell (e.g., about 1 or 2 million HER2 receptors). It is estimated that HER2 is overexpressed (i.e., HER2 IHC3+) in about 15% to about 20% of breast cancers. The cells' expression level of HER2 can be determined by any suitable gene expression technique (e.g., RNA).

Any HER2 amplified cancer is a suitable cancer to be treated by the subject methods and compositions. As used herein, “HER2-amplified cancer” refers to a cell that amplifies the production of the HER2 gene. The amplification of HER2 can be determined by any suitable technique, e.g., by sequencing or in situ hybridization (ISH). In an embodiment, next generation sequencing (NGS) is used. NGS platforms report copy-number variations per their respective algorithm. The cancer cell treated by the methods of the invention can be amplified or not amplified.

The cancer cell can be characterized by immunohistochemical (IHC) staining. The cancer cell treated by the methods of the invention can be IHC0, IHC1+, IHC2+, or IHC3+. If the IHC result is 0 or 1+, the cancer is considered HER2-negative or low, unless the cancer is HER2-gene amplified. If the IHC result is 3+, the cancer is considered HER2-positive. If the IHC result is 2+, the HER2 status of the cancer cell is called “equivocal.” This means that the HER2 status needs to be tested with, for example, ISH or sequencing for HER2-gene amplification to clarify the result. The cancer cell treated by the methods of the invention can be any IHC or ISH level, for example, ISH+, ISH−, IHC1+/ISH+, IHC1+/ISH−, IHC2+/ISH+, or IHC2+/ISH−.

### Modified Fc Region

In some embodiments, the antibodies in the immunoconjugates contain a modified Fc region, wherein the modification modulates the binding of the Fc region to one or more Fc receptors.

The terms “Fe receptor” or “FcR” refer to a receptor that binds to the Fc region of an antibody. There are three main classes of Fc receptors: (1) FcγR which bind to IgG, (2) FcαR which binds to IgA, and (3) FcεR which binds to IgE. The FcγR family includes several members, such as FcγI (CD64), FcγRIIA (CD32A), FcγRIIB (CD32B), FcγRIIIA (CD16A), and FcγRIIIB (CD16B). The Fcγ receptors differ in their affinity for IgG and also have different affinities for the IgG subclasses (e.g., IgG1, IgG2, IgG3, and IgG4).

In some embodiments, the antibodies in the immunoconjugates (e.g., antibodies conjugated to at least two therapeutic agent moieties) contain one or more modifications (e.g., amino acid insertion, deletion, and/or substitution) in the Fc region that results in modulated binding (e.g., increased binding or decreased binding) to one or more Fc receptors (e.g., FcγRI (CD64), FcγRIIA (CD32A), FcγRIIB (CD32B), FcγRIIIA (CD16a), and/or FcγRIIIB (CD16b)) as compared to the native antibody lacking the mutation in the Fc region. In some embodiments, the antibodies in the immunoconjugates contain one or more modifications (e.g., amino acid insertion, deletion, and/or substitution) in the Fc region that reduce the binding of the Fc region of the antibody to FcγRIIB. In some embodiments, the antibodies in the immunoconjugates contain one or more modifications (e.g., amino acid insertion, deletion, and/or substitution) in the Fc region of the antibody that reduce the binding of the antibody to FcγRIIB while maintaining the same binding or having increased binding to FcγRI (CD64), FcγRIIA (CD32A), and/or FcRγIIIA (CD16a) as compared to the native antibody lacking the mutation in the Fc region. In some embodiments, the antibodies in the immunoconjugates contain one of more modifications in the Fc region that increase the binding of the Fc region of the antibody to FcγRIIB.

In some embodiments, the modulated binding is provided by mutations in the Fc region of the antibody relative to the native Fc region of the antibody. The mutations can be in a CH2 domain, a CH3 domain, or a combination thereof. A “native Fc region” is synonymous with a “wild-type Fc region” and comprises an amino acid sequence that is identical to the amino acid sequence of an Fc region found in nature or identical to the amino acid sequence of the Fc region found in the native antibody (e.g., trastuzumab). Native sequence human Fc regions include a native sequence human IgG1 Fc region, native sequence human IgG2 Fc region, native sequence human IgG3 Fc region, and native sequence human IgG4 Fc region, as well as naturally occurring variants thereof. Native sequence Fc includes the various allotypes of Fcs (see, e.g., Jefferis et al., mAbs, 1(4): 332-338 (2009)).

In some embodiments, the mutations in the Fc region that result in modulated binding to one or more Fc receptors can include one or more of the following mutations: SD (S239D), SDIE (S239D/I332E), SE (S267E), SELF (S267E/L328F), SDIE (S239D/I332E), SDIEAL (S239D/I332E/A330L), GA (G236A), ALIE (A330L/I332E), GASDALIE (G236A/S239D/A330L/I332E), V9 (G237D/P238D/P271G/A330R), and V11 (G237D/P238D/H268D/P271G/A330R), and/or one or more mutations at the following amino acids: E233, G237, P238, H268, P271, L328 and A330. Additional Fc region modifications for modulating Fc receptor binding are described in, for example, U.S. Patent Application Publication 2016/0145350 and U.S. Pat. Nos. 7,416,726 and 5,624,821, which are hereby incorporated by reference in their entireties.

In some embodiments, the Fc region of the antibodies of the immunoconjugates are modified to have an altered glycosylation pattern of the Fc region compared to the native non-modified Fc region.

Human immunoglobulin is glycosylated at the Asn297 residue in the Cγ2 domain of each heavy chain. This N-linked oligosaccharide is composed of a core heptasaccharide, (N-acetylglucosamine)4(Mannose)3 (GlcNAc₄Man₃). Removal of the heptasaccharide with endoglycosidase or PNGase F is known to lead to conformational changes in the antibody Fc region, which can significantly reduce antibody-binding affinity to activating FcγR and lead to decreased effector function. The core heptasaccharide is often decorated with galactose, bisecting GlcNAc, fucose, or sialic acid, which differentially impacts Fc binding to activating and inhibitory FcγR. Additionally, it has been demonstrated that α2,6-sialyation enhances anti-inflammatory activity in vivo, while defucosylation leads to improved FcγRIIIa binding and a 10-fold increase in antibody-dependent cellular cytotoxicity and antibody-dependent phagocytosis. Specific glycosylation patterns, therefore, can be used to control inflammatory effector functions.

In some embodiments, the modification to alter the glycosylation pattern is a mutation. For example, a substitution at Asn297. In some embodiments, Asn297 is mutated to glutamine (N297Q). Methods for controlling immune response with antibodies that modulate FcγR-regulated signaling are described, for example, in U.S. Pat. No. 7,416,726 and U.S. Patent Application Publications 2007/0014795 and 2008/0286819, which are hereby incorporated by reference in their entireties.

In some embodiments, the antibodies of the immunoconjugates are modified to contain an engineered Fab region with a non-naturally occurring glycosylation pattern. For example, hybridomas can be genetically engineered to secrete afucosylated mAb, desialylated mAb or deglycosylated Fc with specific mutations that enable increased FcRγIIIa binding and effector function. In some embodiments, the antibodies of the immunoconjugates are engineered to be afucosylated.

In some embodiments, the entire Fc region of an antibody construct of the immunoconjugates is exchanged with a different Fc region, so that the Fab region of the antibody is conjugated to a non-native Fc region. For example, the Fab region of trastuzumab, which normally comprises an IgG1 Fc region, can be conjugated to IgG2, IgG3, IgG4, or IgA, or the Fab region of nivolumab, which normally comprises an IgG4 Fc region, can be conjugated to IgG1, IgG2, IgG3, IgA1, or IgG2. In some embodiments, the Fc modified antibody with a non-native Fc domain also comprises one or more amino acid modification, such as the S228P mutation within the IgG4 Fc, that modulate the stability of the Fc domain described. In some embodiments, the Fc modified antibody with a non-native Fc domain also comprises one or more amino acid modifications described herein that modulate Fc binding to FcR.

In some embodiments, the modifications that modulate the binding of the Fc region to FcR do not alter the binding of the Fab region of the antibody to its antigen when compared to the native non-modified antibody. In other embodiments, the modifications that modulate the binding of the Fc region to FcR also increase the binding of the Fab region of the antibody to its antigen when compared to the native non-modified antibody.

### Immunoconjugate Composition

The invention provides a composition, e.g., a pharmaceutically acceptable composition or formulation, comprising a plurality of immunoconjugates as described herein and optionally a carrier therefor, e.g., a pharmaceutically acceptable carrier. The immunoconjugates can be the same or different in the composition, i.e., the composition can comprise immunoconjugates that have the same number of therapeutic agents conjugated to the same positions on the antibody construct and/or immunoconjugates that have the same number of therapeutic agents conjugated to different positions on the antibody construct, that have different numbers of therapeutic agents conjugated to the same positions on the antibody construct, or that have different numbers of therapeutic agents conjugated to different positions on the antibody construct.

In some embodiments, the composition further comprises one or more pharmaceutically acceptable carrier. For example, the immunoconjugates of the invention can be formulated for parenteral administration, such as IV administration or administration into a body cavity or lumen of an organ. Alternatively, the immunoconjugates can be injected intra-tumorally. Compositions for injection will commonly comprise a solution of the immunoconjugate dissolved in a pharmaceutically acceptable carrier. Among the acceptable vehicles and solvents that can be employed are water and an isotonic solution of one or more salts such as sodium chloride, e.g., Ringer's solution. In addition, sterile fixed oils can conventionally be employed as a solvent or suspending medium. For this purpose, any bland fixed oil can be employed, including synthetic monoglycerides or diglycerides. In addition, fatty acids such as oleic acid can likewise be used in the preparation of injectables. These compositions desirably are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The composition can contain any suitable concentration of the immunoconjugate. The concentration of the immunoconjugate in the composition can vary widely, and will be selected primarily based on fluid volumes, viscosities, body weight, and the like, in accordance with the particular mode of administration selected and the subject's needs. In certain embodiments, the concentration of an immunoconjugate in a solution formulation for injection will range from about 0.1% (w/w) to about 10% (w/w).

### Combination Treatments

An embodiment of the invention provides a method for treating cancer in a subject comprising administering an immunoconjugate of formula: Ab-[TA]ᵣ as described herein, or a pharmaceutically acceptable salt thereof, and further administering an effective amount of an additional (i.e., different) therapy to a subject having cancer. The additional therapy can be any suitable therapy, or any combination of any suitable therapies, many of which are known by those ordinarily skilled in the art, including monitoring the progression of the cancer, surgery, radiation therapy, High Intensity Focused Ultrasound (HIFU), chemotherapy, cryosurgery, hormonal therapy, immunotherapy, targeted monoclonal antibodies, antibody-drug conjugates, tyrosine kinase inhibitors, or a combination thereof. The additional therapy can be consistent with what is considered the standard of care at the time of treatment and/or consistent with the current practices in neoadjuvant, adjuvant 1^{st}-line (1L), 2^{nd}-line (2L), 3^{rd}-line (3L), 4^{th}-line (4L), 5^{th}-line (5L), 6th-line (6L), 7^{th}-line (7L), and beyond treatments for the cancer being treated.

Chemotherapies include administering docetaxel, cabazitaxel, mitoxantrone, estramustine, asparaginase, busulfan, carboplatin, cisplatin, daunorubicin, doxorubicin, fluorouracil, gemcitabine, hydroxyurea, methotrexate, paclitaxel, protein-bound paclitaxel, albumin-bound paclitaxel, vinblastine, capecitabine, eribulin, ixabepilone, liposomal doxorubicin, mitoxantrone, vinorelbine, vincristine, anthracycline, cyclophosphamide, fluorouracil (or 5-fluorouracil or 5-FU), thiotepa, docetaxel, vinorelbine, irinotecan, ixabepilone, temozolamide, topotecan, vincristine, mitomycin, capecitabine, or a combination thereof.

Surgical therapies include removal of the cancer, or a portion thereof. Radiation therapy involves using ionizing radiation, including external beam radiation therapy, CyberKnife therapy, and brachytherapy. Brachytherapy involves implanting small radioactive rods directly into the tumor. Cryosurgery involves inserting metal rods into the cancer and then using argon gas to cool the rods which freezes the surrounding tissue.

Hormonal therapies include the administration of tamoxifen, aromatase inhibitors, orchiectomy, antiandrogens (e.g., ketoconazole, aminoglutethimide, flutamide, bicalutamide, nilutamide, and cyproterone acetate), raloxifene, anastrozole, exemestane, letrozole, leuprolide, buserelin, goserelin, megestrol acetate, risedronate, pamidronate, ibandronate, alendronate, denosumab, zoledronate, toremifene, fulvestrant, and GnRH antagonists (e.g., abarelix).

In an embodiment, the additional therapy is an immunotherapy. Any suitable immunotherapy, or any combination of suitable immunotherapies, is contemplated for use as the additional therapy, such as use of T cell transfers, cancer vaccines, oncolytic viruses, monoclonal antibodies, and immune checkpoint inhibitors.

As used herein, the phrase “immune checkpoint inhibitor” refers to any modulator that inhibits the activity of the immune checkpoint molecule. Immune checkpoint inhibitors can include, but are not limited to, immune checkpoint molecule binding proteins, small molecule inhibitors, antibodies (including bispecific and multispecific antibodies with at least one antigen binding region that targets an immune checkpoint protein, e.g., bispecific or multispecific antibodies that do not exclusively target immune checkpoint proteins, as well as antibodies that are dual immunomodulators (simultaneous targeting two immunomodulating targets), which result in blockade of inhibitory targets, depletion of suppressive cells, and/or activation of effector cells; tumor-targeted immunomodulators (directs potent costimulation to the tumor-infiltrating immune cells by targeting a tumor antigen and costimulatory molecules such as CD40 or 4-1BB); NK-cell redirectors (redirects NK cells to malignant cells by targeting a tumor antigen and CD16A); or T-cell redirectors (redirects T cells to malignant cells by targeting a tumor antigen and CD3)), antibody-derivatives (including Fc fusions, Fab fragments, and scFvs), antibody-drug conjugates, antisense oligonucleotides, siRNA, aptamers, peptides and peptide mimetics.

In some embodiments, the immune checkpoint inhibitor reduces the expression or activity of one or more immune checkpoint proteins. In another embodiment, the immune checkpoint inhibitor reduces the interaction between one or more immune checkpoint proteins and their ligands. Inhibitory nucleic acids that decrease the expression and/or activity of immune checkpoint molecules can also be used in the methods disclosed herein.

In some embodiments, the immune checkpoint inhibitor is cytotoxic T-lymphocyte antigen 4 (CTLA4, also known as CD152), T cell immunoreceptor with Ig and ITIM domains (TIGIT), glucocorticoid-induced TNFR-related protein (GITR, also known as TNFRSF18), inducible T cell costimulatory (ICOS, also known as CD278), CD96, poliovirus receptor-related 2 (PVRL2, also known as CDI 12R), programmed cell death protein 1 (PD-1), programmed cell death ligand 1 (PD-L1), programmed cell death ligand 2 (PD-L2, also known as B7-DC and CD273), lymphocyte activation gene-3 (LAG-3, also known as CD223), B7-H4, killer immunoglobulin receptor (KIR), Tumor Necrosis Factor Receptor superfamily member 4 (TNFRSF4, also known as OX40 and CD134) and its ligand OX40L (CD252), indoleamine 2,3-dioxygenase 1 (IDO-1), indoleamine 2,3-dioxygenase 2 (IDO-2), carcinoembryonic antigen-related cell adhesion molecule 1 (CEACAMI), B and T lymphocyte attenuator (BTLA, also known as CD272), T-cell membrane protein 3 (TIM3), the adenosine A2A receptor (A2Ar), and V-domain Ig suppressor of T cell activation (VISTA protein). In some embodiments, the immune checkpoint inhibitor is an inhibitor of PD-1, PD-L1, or CTLA4.

In some embodiments, the antibody is selected from: ipilimumab (which is available as YERVOY™) pembrolizumab (which is available as KEYTRUDA™) nivolumab (which is available as OPDIVO™), atezolizumab (which is available as TECENTRIQ™), avelumab (which is available as BAVENCIO™), durvalumab (which is available as IMWINZI™), tislelizumab (also referred to as BGB-A317), dostarlimab (also referred to as TSR-042 and WBP-285), and zimberelimab (also referred to as AB122). In some embodiments, the antibody is selected from: ipilimumab, pembrolizumab, nivolumab, atezolizumab, tislelizumab, dostarlimab, and zimberelimab.

In some embodiments, the immune checkpoint inhibitor is an inhibitor of CTLA4, PD-1, PD-L1, PD-L2, LAG-3, B7-H4, KIR, TNFRSF4, OX40L, IDO-1, IDO-2, CEACAMI, BTLA, TIM3, A2Ar, and/or VISTA. In some embodiments, the immune checkpoint inhibitor is an antibody against CTLA4, PD-1, PD-L1, PD-L2, LAG-3, B7-H4, KIR, TNFRSF4, OX40L, IDO-1, IDO-2, CEACAMI, BTLA, TIM3, A2Ar, and/or VISTA. In some embodiments, the immune checkpoint inhibitor is a monoclonal antibody against CTLA4, PD-1, PD-L1, PD-L2, LAG-3, B7-H4, KIR, TNFRSF4, OX40L, IDO-1, IDO-2, CEACAMI, BTLA, TIM3, A2Ar, and/or VISTA. In some embodiments, the immune checkpoint inhibitor is a human or humanized antibody against CTLA4, PD-1, PD-L1, PD-L2, LAG-3, B7-H4, KIR, TNFRSF4, OX40L, IDO-1, IDO-2, CEACAMI, BTLA, TIM3, A2Ar, and/or VISTA. In some embodiments, the immune checkpoint inhibitor reduces the expression or activity of one or more immune checkpoint proteins, such as CTLA4, PD-1, PD-L1, PD-L2, LAG-3, B7-H4, KIR, TNFRSF4, OX40L, IDO-1, IDO-2, CEACAMI, BTLA, TIM3, A2Ar, and/or VISTA. In some embodiments, the immune checkpoint inhibitor reduces the interaction between TNFRSF4 and OX40L. Most checkpoint antibodies are designed not to have effector function as they are not trying to kill cells, but rather to block the signaling.

In an embodiment, the additional therapy is the administration of pertuzumab (which is available as PERJETA™). In a further embodiment, the immunoconjugate as described herein, pertuzumab, and docetaxel are administered to the subject.

In an embodiment, the additional therapy is an antibody-drug conjugate. Antibody-drug conjugates include an antibody linked to a biologically active payload. In some embodiments, the antibody-drug conjugate is DS-8201 (fan-trastuzumab deruxtecan), trastuzumab emtansine, brentuximab vedotin, inotuzumab ozogamicin, gemtuzumab ozogamicin, moxetumomab pasudotox, polatuzumab vedotin-piiq, enfortumab vedotin, belantamab mafodotin-blmf, sacituzumab govitecan, enforumab vedotin, mirvetuximab soravtansine, trastuzumab duocarmazine, anti-folate receptor alpha (FRα) antibody (MOv18-IgG1) conjugated with a Src inhibitor, or a combination thereof.

In an embodiment, the additional therapy is a tyrosine kinase inhibitor. Tyrosine kinase inhibitors are drugs that inhibit tyrosine kinases. In some embodiments, the tyrosine kinase inhibitor is imatinib, gefitinib, erlotinib, dasatinib, sunitinib, adavosertib, tykerb, lapatinib, or a combination thereof.

In an embodiment, the additional therapy is a targeted monoclonal antibody. Targeted monoclonal antibodies are antibodies that target tumor cells. In some embodiments, the targeted monoclonal antibody is an anti-VEGF antibody (e.g., bevacizumab), anti-EGFR antibody (e.g., cetuximab), anti-CD52 antibody (e.g., alemtuzumab), anti-CD20 antibody (e.g., rituximab), anti-HER2 antibody (e.g., trastuzumab and pertuzumab), anti-folate receptor alpha (FRα) antibody (e.g., MOv18-IgG1), anti-TROP2 (also known as epithelial glycoprotein-1, gastrointestinal antigen 733-1, membrane component surface marker-1, tumor-associated calcium signal transducer-2) antibody (e.g., sacituzumab) or a combination thereof. In some embodiments, the targeted monoclonal antibody is a bispecific antibody with at least one antigen binding region that targets tumor cells.

An embodiment of the invention provides a method for treating cancer in a subject comprising administering an immunoconjugate of formula: Ab-[TA]ᵣ as described herein, or a pharmaceutically acceptable salt thereof, and further administering an IgG1 or IgG4 antibody to the subject. In an embodiment, the IgG1 or IgG4 antibody is an anti-PD-1 or an anti-PD-L1 antibody.

PD-L1 (cluster of differentiation 274, CD274, B7-homolog 1, or B7-H1) belongs to the B7 protein superfamily and is a ligand of PD-1 (cluster of differentiation 279, or CD279). The PD-L1/PD-1 axis plays a large role in suppressing the adaptive immune response. More specifically, it is believed that engagement of PD-L1 with its receptor, PD-1, delivers a signal that inhibits activation and proliferation of T-cells. Agents that bind to PD-L1 and prevent the ligand from binding to the PD-1 receptor prevent this immunosuppression and can, therefore, enhance an immune response when desired, such as for the treatment of cancers, autoimmune disorders, or infections.

Several antibodies targeting PD-1 have been developed for the treatment of cancer, including pembrolizumab (which is available as KEYTRUDA™), nivolumab (which is available as OPDIVO™), MEDI0680 (AMP-514), REGN-2810, PDR-001, tislelizumab (BGB-A317), dostarlimab (also referred to as TSR-042 and WBP-285), and zimberelimab (also referred to as AB122). Several antibodies targeting PD-L1 have also been developed for the treatment of cancer, including atezolizumab (which is available as TECENTRIQ™), durvalumab (which is available as IIFINZI™), and avelumab (which is available as BAVENCIO™).

An embodiment of the invention provides an antibody comprising the CDR regions of pembrolizumab. In this regard, the antibody may comprise a first variable region comprising a CDR1 comprising the amino acid sequence of SEQ ID NO: 1 (CDR1 of first variable region), a CDR2 comprising the amino acid sequence of SEQ ID NO: 2 (CDR2 of first variable region), and a CDR3 comprising the amino acid sequence of SEQ ID NO: 3 (CDR3 of first variable region), and a second variable region comprising a CDR1 comprising the amino acid sequence of SEQ ID NO: 4 (CDR1 of second variable region), a CDR2 comprising the amino acid sequence of SEQ ID NO: 5 (CDR2 of second variable region), and a CDR3 comprising the amino acid sequence of SEQ ID NO: 6 (CDR3 of second variable region). In this regard, the antibody can comprise (i) all of SEQ ID NOs: 1-3, (ii) all of SEQ ID NOs: 4-6, or (iii) all of SEQ ID NOs: 1-6. Preferably, the antibody comprises all of SEQ ID NOs: 1-6.

An embodiment of the invention provides an antibody comprising the CDR regions of atezolizumab. In this regard, the antibody may comprise a first variable region comprising a CDR1 comprising the amino acid sequence of SEQ ID NO: 7 (CDR1 of first variable region), a CDR2 comprising the amino acid sequence of SEQ ID NO: 8 (CDR2 of first variable region), and a CDR3 comprising the amino acid sequence of SEQ ID NO: 9 (CDR3 of first variable region), and a second variable region comprising a CDR1 comprising the amino acid sequence of SEQ ID NO: 10 (CDR1 of second variable region), a CDR2 comprising the amino acid sequence of SEQ ID NO: 11 (CDR2 of second variable region), and a CDR3 comprising the amino acid sequence of SEQ ID NO: 12 (CDR3 of second variable region). In this regard, the antibody can comprise (i) all of SEQ ID NOs: 7-9, (ii) all of SEQ ID NOs: 10-12, or (iii) all of SEQ ID NOs: 7-12. Preferably, the antibody comprises all of SEQ ID NOs: 7-12.

An embodiment of the invention provides an antibody comprising the CDR regions of avelumab. In this regard, the antibody may comprise a first variable region comprising a CDR1 comprising the amino acid sequence of SEQ ID NO: 13 (CDR1 of first variable region), a CDR2 comprising the amino acid sequence of SEQ ID NO: 14 (CDR2 of first variable region), and a CDR3 comprising the amino acid sequence of SEQ ID NO: 15 (CDR3 of first variable region), and a second variable region comprising a CDR1 comprising the amino acid sequence of SEQ ID NO: 16 (CDR1 of second variable region), a CDR2 comprising the amino acid sequence of SEQ ID NO: 17 (CDR2 of second variable region), and a CDR3 comprising the amino acid sequence of SEQ ID NO: 18 (CDR3 of second variable region). In this regard, the antibody can comprise (i) all of SEQ ID NOs: 13-15, (ii) all of SEQ ID NOs: 16-18, or (iii) all of SEQ ID NOs: 13-18. Preferably, the antibody comprises all of SEQ ID NOs: 13-18.

An embodiment of the invention provides an antibody comprising the CDR regions of durvalumab. In this regard, the antibody may comprise a first variable region comprising a CDR1 comprising the amino acid sequence of SEQ ID NO: 52 (CDR1 of first variable region), a CDR2 comprising the amino acid sequence of SEQ ID NO: 53 (CDR2 of first variable region), and a CDR3 comprising the amino acid sequence of SEQ ID NO: 54 (CDR3 of first variable region), and a second variable region comprising a CDR1 comprising the amino acid sequence of SEQ ID NO: 55 (CDR1 of second variable region), a CDR2 comprising the amino acid sequence of SEQ ID NO: 56 (CDR2 of second variable region), and a CDR3 comprising the amino acid sequence of SEQ ID NO: 57 (CDR3 of second variable region). In this regard, the antibody can comprise (i) all of SEQ ID NOs: 52-54, (ii) all of SEQ ID NOs: 55-57, or (iii) all of SEQ ID NOs: 52-57. Preferably, the antibody comprises all of SEQ ID NOs: 52-57.

An embodiment of the invention provides an antibody comprising the CDR regions of nivolumab. In this regard, the antibody may comprise a first variable region comprising a CDR1 comprising the amino acid sequence of SEQ ID NO: 58 (CDR1 of first variable region), a CDR2 comprising the amino acid sequence of SEQ ID NO: 59 (CDR2 of first variable region), and a CDR3 comprising the amino acid sequence of SEQ ID NO: 60 (CDR3 of first variable region), and a second variable region comprising a CDR1 comprising the amino acid sequence of SEQ ID NO: 61 (CDR1 of second variable region), a CDR2 comprising the amino acid sequence of SEQ ID NO: 62 (CDR2 of second variable region), and a CDR3 comprising the amino acid sequence of SEQ ID NO: 63 (CDR3 of second variable region). In this regard, the antibody can comprise (i) all of SEQ ID NOs: 58-60, (ii) all of SEQ ID NOs: 61-63, or (iii) all of SEQ ID NOs: 58-63. Preferably, the antibody comprises all of SEQ ID NOs: 58-63.

An embodiment of the invention provides an antibody comprising the CDR regions of tislelizumab. In this regard, the antibody may comprise a first variable region comprising a CDR1 comprising the amino acid sequence of SEQ ID NO: 64 (CDR1 of first variable region), a CDR2 comprising the amino acid sequence of SEQ ID NO: 65 (CDR2 of first variable region), and a CDR3 comprising the amino acid sequence of SEQ ID NO: 66 (CDR3 of first variable region), and a second variable region comprising a CDR1 comprising the amino acid sequence of SEQ ID NO: 67 (CDR1 of second variable region), a CDR2 comprising the amino acid sequence of SEQ ID NO: 68 (CDR2 of second variable region), and a CDR3 comprising the amino acid sequence of SEQ ID NO: 69 (CDR3 of second variable region). In this regard, the antibody can comprise (i) all of SEQ ID NOs: 64-66, (ii) all of SEQ ID NOs: 67-69, or (iii) all of SEQ ID NOs: 64-69. Preferably, the antibody comprises all of SEQ ID NOs: 64-69.

An embodiment of the invention provides an antibody comprising the CDR regions of dostarlimab. In this regard, the antibody may comprise a first variable region comprising a CDR1 comprising the amino acid sequence of SEQ ID NO: 70 (CDR1 of first variable region), a CDR2 comprising the amino acid sequence of SEQ ID NO: 71 (CDR2 of first variable region), and a CDR3 comprising the amino acid sequence of SEQ ID NO: 72 (CDR3 of first variable region), and a second variable region comprising a CDR1 comprising the amino acid sequence of SEQ ID NO: 73 (CDR1 of second variable region), a CDR2 comprising the amino acid sequence of SEQ ID NO: 74 (CDR2 of second variable region), and a CDR3 comprising the amino acid sequence of SEQ ID NO: 75 (CDR3 of second variable region). In this regard, the antibody can comprise (i) all of SEQ ID NOs: 70-72, (ii) all of SEQ ID NOs: 73-75, or (iii) all of SEQ ID NOs: 70-75. Preferably, the antibody comprises all of SEQ ID NOs: 70-75.

An embodiment of the invention provides an antibody comprising the CDR regions of zimberelimab. In this regard, the antibody may comprise a first variable region comprising a CDR1 comprising the amino acid sequence of SEQ ID NO: 76 (CDR1 of first variable region), a CDR2 comprising the amino acid sequence of SEQ ID NO: 77 (CDR2 of first variable region), and a CDR3 comprising the amino acid sequence of SEQ ID NO: 78 (CDR3 of first variable region), and a second variable region comprising a CDR1 comprising the amino acid sequence of SEQ ID NO: 79 (CDR1 of second variable region), a CDR2 comprising the amino acid sequence of SEQ ID NO: 80 (CDR2 of second variable region), and a CDR3 comprising the amino acid sequence of SEQ ID NO: 81 (CDR3 of second variable region). In this regard, the antibody can comprise (i) all of SEQ ID NOs: 76-78, (ii) all of SEQ ID NOs: 79-81, or (iii) all of SEQ ID NOs: 76-81. Preferably, the antibody comprises all of SEQ ID NOs: 76-81.

The methods can include administering the anti-PD-1 or anti-PD-L1 antibody to provide a dose of from about 100 mg to about 2,000 mg to the subject. In this regard, the methods can include administering the anti-PD-1 or anti-PD-L1 antibody to provide a dose of from about 150 mg to about 1,900 mg, from about 175 mg to about 1,800 mg, or from about 190 mg to about 1,700 mg to the subject.

The methods can include administering the anti-PD-1 or anti-PD-L1 antibody to provide a dose of from about 180 mg to about 220 mg to the subject. In this regard, the methods can include administering the anti-PD-1 or anti-PD-L1 antibody to provide a dose of from about 185 mg to about 215 mg, from about 190 mg to about 210 mg, from about 195 mg to about 205 mg, or from about 192 mg to about 202 mg to the subject.

The methods can include administering the anti-PD-1 or anti-PD-L1 antibody to provide a dose of from about 150 mg to about 550 mg to the subject. In this regard, the methods can include administering the anti-PD-1 or anti-PD-L1 antibody to provide a dose of from about 200 mg to about 520 mg, from about 210 mg to about 510 mg, from about 220 mg to about 500 mg, or from about 230 mg to about 490 mg to the subject.

The methods can include administering the anti-PD-1 or anti-PD-L1 antibody to provide a dose of from about 150 mg to about 300 mg to the subject. In this regard, the methods can include administering the anti-PD-1 or anti-PD-L1 antibody to provide a dose of from about 200 mg to about 280 mg, from about 210 mg to about 270 mg, from about 220 mg to about 260 mg, or from about 230 mg to about 250 mg to the subject.

The methods can include administering the anti-PD-1 or anti-PD-L1 antibody to provide a dose of from about 380 mg to about 420 mg to the subject. In this regard, the methods can include administering the anti-PD-1 or anti-PD-L1 antibody to provide a dose of from about 385 mg to about 415 mg, from about 390 mg to about 410 mg, from about 395 mg to about 405 mg, or from about 398 mg to about 402 mg to the subject.

The methods can include administering the anti-PD-1 or anti-PD-L1 antibody to provide a dose of from about 400 mg to about 550 mg to the subject. In this regard, the methods can include administering the anti-PD-1 or anti-PD-L1 antibody to provide a dose of from about 440 mg to about 520 mg, from about 450 mg to about 510 mg, from about 460 mg to about 500 mg, or from about 470 mg to about 490 mg to the subject.

The methods can include administering the anti-PD-1 or anti-PD-L1 antibody to provide a dose of from about 750 mg to about 900 mg to the subject. In this regard, the methods can include administering the anti-PD-1 or anti-PD-L1 antibody to provide a dose of from about 800 mg to about 880 mg, from about 810 mg to about 870 mg, from about 820 mg to about 860 mg, or from about 830 mg to about 850 mg to the subject.

The methods can include administering the anti-PD-1 or anti-PD-L1 antibody to provide a dose of from about 750 mg to about 850 mg to the subject. In this regard, the methods can include administering the anti-PD-1 or anti-PD-L1 antibody to provide a dose of from about 760 mg to about 840 mg, from about 770 mg to about 830 mg, from about 780 mg to about 820 mg, or from about 790 mg to about 810 mg to the subject.

The methods can include administering the anti-PD-1 or anti-PD-L1 antibody to provide a dose of from about 1,100 mg to about 1,300 mg to the subject. In this regard, the methods can include administering the anti-PD-1 or anti-PD-L1 antibody to provide a dose of from about 1,160 mg to about 1,240 mg, from about 1,170 mg to about 1,230 mg, from about 1,180 mg to about 1,220 mg, or from about 1,190 mg to about 1,210 mg to the subject.

The methods can include administering the anti-PD-1 or anti-PD-L1 antibody to provide a dose of from about 1,600 mg to about 1,750 mg to the subject. In this regard, the methods can include administering the anti-PD-1 or anti-PD-L1 antibody to provide a dose of from about 1,640 mg to about 1,720 mg, from about 1,650 mg to about 1,710 mg, from about 1,660 mg to about 1,700 mg, or from about 1,670 mg to about 1,690 mg to the subject.

The methods can include administering the anti-PD-1 or anti-PD-L1 antibody to provide a dose of from about 1 mg/kg to about 50 mg/kg to the subject (e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, and about 50 mg/kg). In this regard, the methods can include administering the anti-PD-1 or anti-PD-L1 antibody to provide a dose of from about 1 mg/kg to about 25 mg/kg, about 3 mg/kg to about 25 mg/kg, from about 5 mg/kg to about 15 mg/kg, from about 7 mg/kg to about 13 mg/kg, from about 9 mg/kg to about 11 mg/kg, from about 1 mg/kg to about 3 mg/kg, from about 1.5 mg/kg to about 2.5 mg/kg, from about 4 mg/kg to about 6 mg/kg, from about 4.5 mg/kg to about 5.5 mg/kg, from about 7 mg/kg to about 9 mg/kg, from about 7.5 mg/kg to about 8.5 mg/kg, from about 11 mg/kg to about 13 mg/kg, from about 11.5 mg/kg to about 12.5 mg/kg, about 2 mg/kg, about 5 mg/kg, about 8 mg/kg, or about 12 mg/kg to the subject.

In some embodiments, the anti-PD-1 or anti-PD-L1 antibody is administered from about every 7 to about every 45 days (e.g., about every 3 days, about every 4 days, about every 5 days, about every 6 days, about every 7 days, about every 8 days, about every 9 days, about every 10 days, about every 11 days, about every 12 days, about every 13 days, about every 14 days, about every 15 days, about every 16 days, about every 17 days, about every 18 days, about every 19 days, about every 20 days, about every 21 days, about every 22 days, about every 23 days, about every 24 days, about every 25 days, about every 26 days, about every 27 days, about every 28 days, about every 29 days, about every 30 days, about every 31 days, about every 32 days, about every 33 days, about every 34 days, about every 35 days, about every 36 days, about every 37 days, about every 38 days, about every 39 days, about every 40 days, about every 41 days, about every 42 days, about every 43 days, about every 44 days, or about every 45 days). In some embodiments, the anti-PD-1 or anti-PD-L1 antibody is administered from about every 3 to about every 35 days. In some embodiments, the anti-PD-1 or anti-PD-L1 antibody is administered every 1, 2, 3, 4, 5, 6, or 7 weeks, or every month. In some embodiments, the anti-PD-1 or anti-PD-L1 antibody is administered from about every 9 to about every 33 days, from about every 11 to about every 31 days, from about every 13 to about every 29 days, from about every 15 to about every 27 days, from about every 17 to about every 25 days, or from about every 19 to about every 23 days. In some embodiments, the anti-PD-1 or anti-PD-L1 antibody is administered every 14 days. In some embodiments, the anti-PD-1 or anti-PD-L1 antibody is administered every 21 days. In some embodiments, the anti-PD-1 or anti-PD-L1 antibody is administered every 28 days. In some embodiments, the anti-PD-1 or anti-PD-L1 antibody is administered every 35 days. In some embodiments, the anti-PD-1 or anti-PD-L1 antibody is administered every 42 days.

In some embodiments, the anti-PD-1 or anti-PD-L1 antibody is administered from about every 5 to about every 9 days, from about every 6 to about every 8 days, from about every 13 to about every 15 days, from about every 12 to about every 16 days, from about every 20 to about every 22 days, from about every 19 to about every 23 days, from about every 27 to about every 29 days, from about every 26 to about every 30 days, from about every 33 to about every 37 days, or from about every 40 days to about every 44 days. In some embodiments, the anti-PD-1 or anti-PD-L1 antibody is administered about every 7 days, about every about 14 days, about every 21 days, about every 28 days, about every 30 days, about every 35 days, or about every 42 days.

The anti-PD-1 or anti-PD-L1 antibody can be administered to the subject using any suitable means including parenteral, intravenous, intraperitoneal, intramuscular, intratumoral, intralesional, intranasal, or subcutaneous administration, oral administration, administration as a suppository, topical contact, intrathecal administration, or the implantation of a slow-release device, e.g., a mini-osmotic pump, to the subject.

In some embodiments, the anti-PD-1 or anti-PD-L1 antibody is administered subcutaneously.

In some embodiments, the anti-PD-1 or anti-PD-L1 antibody is administered intravenously (IV). In some embodiments, the anti-PD-1 or anti-PD-L1 antibody is administered via IV infusion. In some embodiments, the anti-PD-1 or anti-PD-L1 antibody is administered to the subject intravenously over about 1 to about 60 minutes. In this regard, the anti-PD-1 or anti-PD-L1 antibody is administered over about 5 to about 55 minutes, over about 10 to about 50 minutes, over about 15 to about 45 minutes, over about 20 to about 40 minutes, over about 25 to about 35 minutes, or over about 30 minutes to the subject.

In some embodiments, the additional therapy (e.g., the standard of care and across various lines of therapy (e.g., adjuvant, neoadjuvant, 1L, 2L, 3L, 4L, 5L, 6L, 7L, and beyond for the indication, e.g., chemotherapy, the anti-PD-1 antibody, anti-PD-L1 antibody, or pertuzumab)) is administered concurrently with the immunoconjugate to the subject. In another embodiment, the additional therapy (e.g., the standard of care and across various lines of therapy (e.g., adjuvant, neoadjuvant, 1L, 2L, 3L, 4L, 5L, 6L, 7L, and beyond for the indication, e.g., chemotherapy, the anti-PD-1 antibody, anti-PD-L1 antibody, or pertuzumab)) can be administered sequentially to the subject.

As used herein, the phrases “concurrent administration” or “concurrently” or “simultaneous” mean that administration of the immunoconjugate and the additional therapy (e.g., the standard of care and across various lines of therapy (e.g., adjuvant, neoadjuvant, 1L, 2L, 3L, 4L, 5L, 6L, 7L, and beyond for the indication, e.g., chemotherapy, the anti-PD-1 antibody, anti-PD-L1 antibody, or pertuzumab)) occurs on the same day. The terms “sequential administration,” “sequentially,” or “separate” mean that administration occurs on different days.

“Simultaneous” administration, as defined herein, includes the administration of the immunoconjugate and the additional therapy (e.g., the standard of care and across various lines of therapy (e.g., adjuvant, neoadjuvant, 1L, 2L, 3L, 4L, 5L, 6L, 7L, and beyond for the indication, e.g., chemotherapy, the anti-PD-1 antibody, anti-PD-L1 antibody, or pertuzumab)) within about 2 hours or about 1 hour or less of each other, even more preferably at the same time.

“Separate” administration, as defined herein, includes the administration of the immunoconjugate and the additional therapy (e.g., the standard of care and across various lines of therapy (e.g., adjuvant, neoadjuvant, 1L, 2L, 3L, 4L, 5L, 6L, 7L, and beyond for the indication, e.g., chemotherapy, the anti-PD-1 antibody, anti-PD-L1 antibody, or pertuzumab)), more than about 12 hours, or about 8 hours, or about 6 hours or about 4 hours or about 2 hours apart.

“Sequential” administration, as defined herein, includes the administration of the immunoconjugate and the additional therapy (e.g., the standard of care and across various lines of therapy (e.g., adjuvant, neoadjuvant, 1L, 2L, 3L, 4L, 5L, 6L, 7L, and beyond for the indication, e.g., chemotherapy, the anti-PD-1 antibody, anti-PD-L1 antibody, or pertuzumab)) each in multiple aliquots and/or doses and/or on separate occasions. The immunoconjugate may be administered to the subject before and/or after administration of the additional therapy (e.g., the standard of care and across various lines of therapy (e.g., adjuvant, neoadjuvant, 1L, 2L, 3L, 4L, 5L, 6L, 7L, and beyond for the indication, e.g., chemotherapy, the anti-PD-1 antibody, anti-PD-L1 antibody, or pertuzumab)).

The additional therapy can be administered to the subject as an initial loading dose followed by one or more maintenance doses. The loading dose of the additional therapy may be a higher or lower dose than the one or more maintenance doses. The loading dose of the additional therapy may be administered to the patient using a similar or different suitable means than the one or more maintenance doses.

The immunoconjugate, or a pharmaceutically acceptable salt thereof, can be administered to the subject at about 0.15 mg/kg in combination with concurrent administration of pembrolizumab at about 200 mg about every 3 weeks by IV infusion. The immunoconjugate, or a pharmaceutically acceptable salt thereof, can be administered to the subject at about 0.5 mg/kg in combination with concurrent administration of pembrolizumab at about 200 about every 3 weeks by IV infusion. The immunoconjugate, or a pharmaceutically acceptable salt thereof, can be administered to the subject at about 2 mg/kg in combination with concurrent administration of pembrolizumab at about 200 mg about every 3 weeks by IV infusion. The immunoconjugate, or a pharmaceutically acceptable salt thereof, can be administered to the subject at about 5 mg/kg in combination with concurrent administration of pembrolizumab at about 200 mg about every 3 weeks by IV infusion. The immunoconjugate, or a pharmaceutically acceptable salt thereof, can be administered to the subject at about 8 mg/kg in combination with concurrent administration of pembrolizumab at about 200 mg about every 3 weeks by IV infusion. The immunoconjugate, or a pharmaceutically acceptable salt thereof, can be administered to the subject at about 12 mg/kg in combination with concurrent administration of pembrolizumab at about 200 mg about every 3 weeks by IV infusion. The immunoconjugate, or a pharmaceutically acceptable salt thereof, can be administered to the subject at 20 mg/kg in combination with concurrent administration of pembrolizumab at about 200 mg about every 3 weeks by IV infusion.

The immunoconjugate, or a pharmaceutically acceptable salt thereof, can be administered to the subject at about 0.15 mg/kg in combination with concurrent administration of pembrolizumab at about 400 mg about every 6 weeks by IV infusion. The immunoconjugate, or a pharmaceutically acceptable salt thereof, can be administered to the subject at about 5 mg/kg in combination with concurrent administration of pembrolizumab at about 400 mg about every 6 weeks by IV infusion. The immunoconjugate, or a pharmaceutically acceptable salt thereof, can be administered to the subject at about 8 mg/kg in combination with concurrent administration of pembrolizumab at about 400 mg about every 6 weeks by IV infusion. The immunoconjugate, or a pharmaceutically acceptable salt thereof, can be administered to the subject at about 12 mg/kg in combination with concurrent administration of pembrolizumab at about 400 mg about every 6 weeks by IV infusion. The immunoconjugate, or a pharmaceutically acceptable salt thereof, can be administered to the subject at 20 mg/kg in combination with concurrent administration of pembrolizumab at about 400 mg about every 6 weeks by IV infusion.

The immunoconjugate, or a pharmaceutically acceptable salt thereof, can be administered to the subject at about 0.15 mg/kg in combination with concurrent administration of nivolumab at about 240 mg about every 2 weeks by IV infusion. The immunoconjugate, or a pharmaceutically acceptable salt thereof, can be administered to the subject at about 5 mg/kg in combination with concurrent administration of nivolumab at about 240 mg about every 2 weeks by IV infusion. The immunoconjugate, or a pharmaceutically acceptable salt thereof, can be administered to the subject at about 8 mg/kg in combination with concurrent administration of nivolumab at about 240 mg about every 2 weeks by IV infusion. The immunoconjugate, or a pharmaceutically acceptable salt thereof, can be administered to the subject at about 12 mg/kg in combination with concurrent administration of nivolumab at about 240 mg about every 2 weeks by IV infusion. The immunoconjugate, or a pharmaceutically acceptable salt thereof, can be administered to the subject at 20 mg/kg in combination with concurrent administration of nivolumab at about 240 mg about every 2 weeks by IV infusion.

The immunoconjugate, or a pharmaceutically acceptable salt thereof, can be administered to the subject at about 0.15 mg/kg in combination with concurrent administration of nivolumab at about 480 mg about every 4 weeks by IV infusion. The immunoconjugate, or a pharmaceutically acceptable salt thereof, can be administered to the subject at about 5 mg/kg in combination with concurrent administration of nivolumab at about 480 mg about every 4 weeks by IV infusion. The immunoconjugate, or a pharmaceutically acceptable salt thereof, can be administered to the subject at about 8 mg/kg in combination with concurrent administration of nivolumab at about 480 mg about every 4 weeks by IV infusion. The immunoconjugate, or a pharmaceutically acceptable salt thereof, can be administered to the subject at about 12 mg/kg in combination with concurrent administration of nivolumab at about 480 mg about every 4 weeks by IV infusion. The immunoconjugate, or a pharmaceutically acceptable salt thereof, can be administered to the subject at 20 mg/kg in combination with concurrent administration of nivolumab at about 480 mg about every 4 weeks by IV infusion.

The immunoconjugate, or a pharmaceutically acceptable salt thereof, can be administered to the subject at about 0.15 mg/kg in combination with concurrent administration of atezolizumab at about 840 mg about every 2 weeks by IV infusion. The immunoconjugate, or a pharmaceutically acceptable salt thereof, can be administered to the subject at about 5 mg/kg in combination with concurrent administration of atezolizumab at about 840 mg about every 2 weeks by IV infusion. The immunoconjugate, or a pharmaceutically acceptable salt thereof, can be administered to the subject at about 8 mg/kg in combination with concurrent administration of atezolizumab at about 840 mg about every 2 weeks by IV infusion. The immunoconjugate, or a pharmaceutically acceptable salt thereof, can be administered to the subject at about 12 mg/kg in combination with concurrent administration of atezolizumab at about 840 mg about every 2 weeks by IV infusion. The immunoconjugate, or a pharmaceutically acceptable salt thereof, can be administered to the subject at 20 mg/kg in combination with concurrent administration of atezolizumab at about 840 mg about every 2 weeks by IV infusion.

The immunoconjugate, or a pharmaceutically acceptable salt thereof, can be administered to the subject at about 0.15 mg/kg in combination with concurrent administration of atezolizumab at about 1200 mg about every 3 weeks by IV infusion. The immunoconjugate, or a pharmaceutically acceptable salt thereof, can be administered to the subject at about 5 mg/kg in combination with concurrent administration of atezolizumab at about 1200 mg about every 3 weeks by IV infusion. The immunoconjugate, or a pharmaceutically acceptable salt thereof, can be administered to the subject at about 8 mg/kg in combination with concurrent administration of atezolizumab at about 1200 mg about every 3 weeks by IV infusion. The immunoconjugate, or a pharmaceutically acceptable salt thereof, can be administered to the subject at about 12 mg/kg in combination with concurrent administration of atezolizumab at about 1200 mg about every 3 weeks by IV infusion. The immunoconjugate, or a pharmaceutically acceptable salt thereof, can be administered to the subject at 20 mg/kg in combination with concurrent administration of atezolizumab at about 1200 mg about every 3 weeks by IV infusion.

The immunoconjugate, or a pharmaceutically acceptable salt thereof, can be administered to the subject at about 0.15 mg/kg in combination with concurrent administration of atezolizumab at about 1680 mg about every 4 weeks by IV infusion. The immunoconjugate, or a pharmaceutically acceptable salt thereof, can be administered to the subject at about 5 mg/kg in combination with concurrent administration of atezolizumab at about 1680 mg about every 4 weeks by IV infusion. The immunoconjugate, or a pharmaceutically acceptable salt thereof, can be administered to the subject at about 8 mg/kg in combination with concurrent administration of atezolizumab at about 1680 mg about every 4 weeks by IV infusion. The immunoconjugate, or a pharmaceutically acceptable salt thereof, can be administered to the subject at about 12 mg/kg in combination with concurrent administration of atezolizumab at about 1680 mg about every 4 weeks by IV infusion. The immunoconjugate, or a pharmaceutically acceptable salt thereof, can be administered to the subject at 20 mg/kg in combination with concurrent administration of atezolizumab at about 1680 mg about every 4 weeks by IV infusion.

### Treatment and Prevention

The invention provides a method for treating cancer. The method includes comprising administering an immunoconjugate, or a pharmaceutically acceptable salt thereof, as described herein (e.g., as a composition as described herein), alone or as part of a combination treatment as described herein, to a subject in need thereof, e.g., a subject that has cancer and is in need of treatment for the cancer.

Trastuzumab and pertuzumab, biosimilars thereof, and biobetters thereof are known to be useful in the treatment of cancer, particularly breast cancer, especially HER2-overexpressing breast cancer, gastric cancer, especially HER2-overexpressing gastric cancer, and gastroesophageal junction adenocarcinoma. The immunoconjugate, or pharmaceutically acceptable salt thereof, described herein, alone or as part of a combination treatment as described herein, can be used to treat the same types of cancers as trastuzumab, pertuzumab, biosimilars thereof, and biobetters thereof particularly breast cancer, especially HER2-overexpressing breast cancer, gastric cancer, especially HER2-overexpressing gastric cancer, gastroesophageal junction adenocarcinoma, lung cancer, endometrial cancer, colorectal cancer, and salivary gland cancer.

Some embodiments of the invention provide methods for treating cancer as described above, wherein the cancer is breast cancer. Breast cancer can originate from different areas in the breast, and a number of different types of breast cancer have been characterized. For example, the immunoconjugates of the invention can be used for treating ductal carcinoma in situ; invasive ductal carcinoma (e.g., tubular carcinoma; medullary carcinoma; mucinous carcinoma; papillary carcinoma; or cribriform carcinoma of the breast); lobular carcinoma in situ; invasive lobular carcinoma; inflammatory breast cancer; and other forms of breast cancer.

Some embodiments of the invention provide methods for treating cancer as described above, wherein the cancer is gastric cancer. Gastric (stomach) cancer can originate from different cells in the stomach and several types of gastric cancer have been characterized including adenocarcinoma, carcinoid tumors, squamous cell carcinoma, small cell carcinoma, leiomyosarcoma, and gastrointestinal stromal tumors.

Some embodiments of the invention provide methods for treating cancer as described above, wherein the cancer is gastroesophageal junction carcinoma. This carcinoma occurs in the area where the esophagus meats the stomach. There are three types of gastroesophageal junction carcinoma. In Type 1, the cancer the cancer grows down from above and into the gastroesophageal junction. The normal lining of the lower end of the esophagus is replaced by mutations (also called Barrett's esophagus). In Type 2, the cancer grows at the gastroesophageal junction by itself. In Type 3, the cancer grows up into the gastroesophageal junction from the stomach upwards.

Some embodiments of the invention provide methods for treating cancer as described above, wherein the cancer is colorectal. This carcinoma occurs in the colon and/or rectum. The most common type of colorectal cancer is adenocarcinoma. Other types of colorectal cancer include adenosquamous and squamous cell carcinoma.

Some embodiments of the invention provide methods for treating cancer as described above, wherein the cancer is lung cancer. Lung cancer begins in the lungs. Types of lung cancer include small cell lung cancer and non-small cell lung cancers. Non-small cell lung cancers include adenocarcinoma, squamous cell carcinoma, and large cell carcinoma.

Some embodiments of the invention provide methods for treating cancer as described above, wherein the cancer has metastasized.

Some embodiments of the invention provide methods for treating cancer as described above, wherein the cancer is endometrial. This carcinoma occurs in the layer of cells that form the lining (endometrium) of the uterus. Types of endometrial cancer include edenocarcinoma, uterine carcinosarcoma, squamous cell carcinoma, small cell carcinoma, transitional carcinoma, and serous carcinoma.

Some embodiments of the invention provide methods for treating cancer as described above, wherein the cancer is salivary gland. This carcinoma occurs in salivary gland. Types of salivary gland cancer include acinic cell carcinoma, adenocarcinoma, adenoid cystic carcinoma, clear cell carcinoma, malignant mixed tumor, mucoepidermoid carcinoma, oncocytic carcinoma, polymorphous low-grade adenocarcinoma, salivary duct carcinoma, and squamous cell carcinoma.

Some embodiments of the invention provide methods for treating cancer in a subject. In an embodiment, the subject is a human.

### Aspects of the Disclosure

Aspects, including embodiments, of the invention described herein may be beneficial alone or in combination, with one or more other aspects or embodiments. Without limiting the foregoing description, certain non-limiting aspects of the disclosure numbered 1-49 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) A method for treating cancer in a subject comprising administering from about 0.01 to about 100 mg/kg of an immunoconjugate of formula: Ab-[TA]ᵣ or a pharmaceutically acceptable salt thereof, wherein “Ab” is an antibody construct that has an antigen binding domain that binds human epidermal growth factor receptor type 2 (HER2) and “TA” is a therapeutic agent of formula:

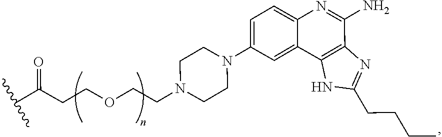

wherein n is from about 2 to about 25 and r is an average therapeutic agent to antibody ratio from 1 to 10, to a subject having cancer.

(2) A method for treating cancer in a subject comprising administering from about every 3 to about every 45 days an immunoconjugate of formula: Ab-[TA]ᵣ or a pharmaceutically acceptable salt thereof, wherein “Ab” is an antibody construct that has an antigen binding domain that binds human epidermal growth factor receptor type 2 (HER2) and “TA” is a therapeutic agent of formula:

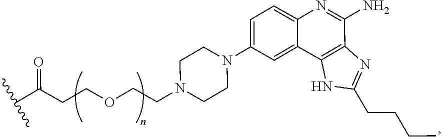

wherein n is from about 2 to about 25 and r is an average therapeutic agent to antibody ratio from about 1 to about 10, to a subject having cancer.

(3) The method of aspect 2, wherein from about 0.01 to about 100 mg/kg of an immunoconjugate is administered to the subject during each administration.

(4) The method of any one of aspects 1-3, wherein the immunoconjugate is administered in the form of a composition comprising the immunoconjugate and a pharmaceutically acceptable carrier therefor.

(5) The method of any one of aspects 1-4, wherein the immunoconjugate is administered to the subject intravenously.

(6) The method of aspect 5, wherein the immunoconjugate is administered to the subject intravenously over about 1 to about 240 minutes.

(7) The method of any one of aspects 1-6, further comprising administering an effective amount of an additional therapy to the subject having cancer.

(8) The method of aspect 7, wherein the additional therapy is selected from the group consisting of surgery, radiation therapy, High Intensity Focused Ultrasound (HIFU), chemotherapy, cryosurgery, hormonal therapy, immunotherapy, targeted monoclonal antibodies, antibody-drug conjugates, tyrosine kinase inhibitors, or a combination thereof.

(9) The method of aspect 7, wherein the additional therapy is an immunotherapy.

(10) The method of any one of aspects 7-9, wherein the additional therapy is an immune checkpoint inhibitor.

(11) The method of any one of aspects 7-10, wherein the additional therapy is an IgG1 or IgG4 antibody.

(12) The method of aspect 11, wherein the IgG1 or IgG4 antibody is an anti-programmed cell death protein 1 (PD-1) or an anti-programmed death-ligand 1 (PD-L1) antibody.

(13) The method of aspect 12, wherein the antibody is an anti-PD-1 antibody.

(14) The method of aspect 13, wherein the anti-PD-1 antibody comprises a variable light (VL) chain region comprising a CDR1 having the amino acid sequence of SEQ ID NO: 1, a CDR2 having the amino acid sequence of SEQ ID NO: 2, and a CDR3 having the amino acid sequence of SEQ ID NO: 3; and a variable heavy (VH) chain region comprising a CDR1 having the amino acid sequence of SEQ ID NO: 4, a CDR2 having the amino acid sequence of SEQ ID NO: 5, and a CDR3 having the amino acid sequence of SEQ ID NO: 6.

(15) The method of aspect 13, wherein the anti-PD-1 antibody comprises a variable light (VL) chain region comprising a CDR1 having the amino acid sequence of SEQ ID NO: 58, a CDR2 having the amino acid sequence of SEQ ID NO: 59, and a CDR3 having the amino acid sequence of SEQ ID NO: 60; and a variable heavy (VH) chain region comprising a CDR1 having the amino acid sequence of SEQ ID NO: 61, a CDR2 having the amino acid sequence of SEQ ID NO: 62, and a CDR3 having the amino acid sequence of SEQ ID NO: 63.

(16) The method of aspect 13, wherein the anti-PD-1 antibody comprises a variable light (VL) chain region comprising a CDR1 having the amino acid sequence of SEQ ID NO: 64, a CDR2 having the amino acid sequence of SEQ ID NO: 65, and a CDR3 having the amino acid sequence of SEQ ID NO: 66; and a variable heavy (VH) chain region comprising a CDR1 having the amino acid sequence of SEQ ID NO: 67, a CDR2 having the amino acid sequence of SEQ ID NO: 68, and a CDR3 having the amino acid sequence of SEQ ID NO: 69.

(17) The method of aspect 13, wherein the anti-PD-1 antibody comprises a variable light (VL) chain region comprising a CDR1 having the amino acid sequence of SEQ ID NO: 70, a CDR2 having the amino acid sequence of SEQ ID NO: 71, and a CDR3 having the amino acid sequence of SEQ ID NO: 72; and a variable heavy (VH) chain region comprising a CDR1 having the amino acid sequence of SEQ ID NO: 73, a CDR2 having the amino acid sequence of SEQ ID NO: 74, and a CDR3 having the amino acid sequence of SEQ ID NO: 75.

(18) The method of aspect 13, wherein the anti-PD-1 antibody comprises a variable light (VL) chain region comprising a CDR1 having the amino acid sequence of SEQ ID NO: 76, a CDR2 having the amino acid sequence of SEQ ID NO: 77, and a CDR3 having the amino acid sequence of SEQ ID NO: 78; and a variable heavy (VH) chain region comprising a CDR1 having the amino acid sequence of SEQ ID NO: 79, a CDR2 having the amino acid sequence of SEQ ID NO: 80, and a CDR3 having the amino acid sequence of SEQ ID NO: 81.

(19) The method of aspect 12, wherein the antibody is an anti-PD-L1 antibody.

(20) The method of aspect 19, wherein the anti-PD-L1 antibody comprises a variable light (VL) chain region comprising a CDR1 having the amino acid sequence of SEQ ID NO: 7, a CDR2 having the amino acid sequence of SEQ ID NO: 8, and a CDR3 having the amino acid sequence of SEQ ID NO: 9; and a variable heavy (VH) chain region comprising a CDR1 having the amino acid sequence of SEQ ID NO: 10, a CDR2 having the amino acid sequence of SEQ ID NO: 11, and a CDR3 having the amino acid sequence of SEQ ID NO: 12.

(21) The method of aspect 19, wherein the anti-PD-L1 antibody comprises a variable light (VL) chain region comprising a CDR1 having the amino acid sequence of SEQ ID NO: 13, a CDR2 having the amino acid sequence of SEQ ID NO: 14, and a CDR3 having the amino acid sequence of SEQ ID NO: 15; and a variable heavy (VH) chain region comprising a CDR1 having the amino acid sequence of SEQ ID NO: 16, a CDR2 having the amino acid sequence of SEQ ID NO: 17, and a CDR3 having the amino acid sequence of SEQ ID NO: 18.

(22) The method of any one of aspects 12-21, wherein from about 100 mg to about 2,000 mg of the antibody is administered to the subject.

(23) The method of any one of aspects 12-21, wherein the antibody is administered to the subject intravenously.

(24) The method of aspect 23, wherein the antibody is administered to the subject intravenously over about 1 minute to about 60 minutes.

(25) The method of any one of aspects 12-24, wherein the immunoconjugate is concurrently administered with the antibody.

(26) The method of any one of aspects 12-25, wherein the antibody is administered from about every 7 days to about every 45 days.

(27) The method of any one of aspects 1-26, wherein the cancer is a HER2-expressing or HER2-amplified cancer.

(28) The method of any one of aspects 1-27, wherein the cancer is breast cancer.

(29) The method of aspect 28, wherein the cancer is HER2 overexpressing breast cancer.

(30) The method of any one of aspects 1-27, wherein the cancer is gastric cancer.

(31) The method of aspect 30, wherein the cancer is HER2 overexpressing gastric cancer.

(32) The method of any one of aspects 1-27, wherein the cancer is gastroesophageal junction adenocarcinoma.

(33) The method of any one of aspects 1-27, wherein the cancer is colorectal cancer.

(34) The method of any one of aspects 1-27, wherein the cancer is endometrial cancer.

(35) The method of any one of aspects 1-27, wherein the cancer is salivary gland cancer.

(36) The method of any one of aspects 1-27, wherein the cancer is lung cancer.

(37) The method of any one of aspects 1-36, wherein the cancer has metastasized.

(38) The method of any one of aspects 1-37, wherein the cancer is HER2 IHC1+/ISH+.

(39) The method of any one of aspects 1-37, wherein the cancer is HER2 IHC1+/ISH−.

(40) The method of any one of aspects 1-37, wherein the cancer is HER2 IHC2+/ISH+.

(41) The method of any one of aspects 1-37, wherein the cancer is HER2 IHC2+/ISH−.

(42) The method of any one of aspects 1-37, wherein the cancer is HER2 IHC3+.

(43) The method of any one of aspects 1-42, wherein the cancer is expressing or over-expressing HER2 as determined by gene expression.

(44) The method of any one of aspects 1-42, wherein the cancer exhibits HER2 amplification.

(45) The method of aspect 44, wherein the cancer is ISH+.

(46) The method of aspect 44, wherein the cancer is ISH−.

(47) The method of any one of aspects 44-46, wherein the HER2 amplification is determined by sequencing.

(48) The method of any one of aspects 44-46, wherein the HER2 amplification is determined by next generation sequencing (NGS).

(49) The method of any one of aspects 1-48, wherein r is from about 1 to about 6.

(50) The method of aspect 49, wherein r is from about 2 to about 4.

(51) The method of any one of aspects 1-50, wherein n is from about 6 to about 12.

(52) The method of aspect 51, wherein n is about 10.

(53) The method of any one of aspects 1-52, wherein “Ab” is trastuzumab, a biosimilar thereof, or a biobetter thereof.

(54) The method of any one of aspects 1-52, wherein “Ab” is pertuzumab, a biosimilar thereof, or a biobetter thereof.

(55) The method of any one of aspects 1-52, wherein “Ab” is trastuzumab.

(56) The method of any one of aspects 1-52, wherein “Ab” is a biosimilar of trastuzumab.

(57) The method of any one of claims 1-52, wherein “Ab” is a biosimilar of pertuzumab.

(58) The method of any one of aspects 1-57, wherein the subject is treated for from about 1 month to about 48 months.

(59) The method of any one of aspects 1-58, wherein the subject is human.

(60) An immunoconjugate of formula: Ab-[TA]ᵣ or a pharmaceutically acceptable salt thereof, wherein “Ab” is an antibody construct that has an antigen binding domain that binds human epidermal growth factor receptor type 2 (HER2) and “TA” is a therapeutic agent of formula:

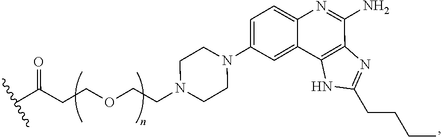

wherein n is from about 2 to about 25 and r is an average therapeutic agent to antibody ratio from about 1 to about 10, for use as a medicament for treating cancer, wherein from about 0.01 to about 100 mg/kg of the immunoconjugate or a pharmaceutically acceptable salt thereof is administered to a subject having cancer.

(61) An immunoconjugate of formula: Ab-[TA]ᵣ or a pharmaceutically acceptable salt thereof, wherein “Ab” is an antibody construct that has an antigen binding domain that binds human epidermal growth factor receptor type 2 (HER2) and “TA” is a therapeutic agent of formula:

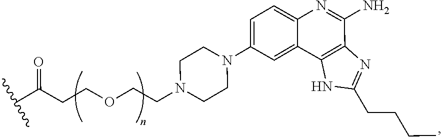

wherein n is from about 2 to about 25 and r is an average therapeutic agent to antibody ratio from 1 to 10, for use as a medicament for treating cancer, wherein the immunoconjugate or a pharmaceutically acceptable salt thereof is administered from about every 3 to about every 45 days to a subject having cancer.

(62) The use of aspect 61, wherein from about 0.01 to about 100 mg/kg of an immunoconjugate is administered to the subject during each administration.

(63) The use of any one of aspects 60-62, wherein the immunoconjugate is administered in the form of a composition comprising the immunoconjugate and a pharmaceutically acceptable carrier therefor.

(64) The use of any one of aspects 60-63, wherein the immunoconjugate is administered to the subject intravenously.

(65) The use of aspect 64, wherein the immunoconjugate is administered to the subject intravenously over about 1 to about 240 minutes.

(66) The use of any one of aspects 60-65, further comprising administering an effective amount of an additional therapy to the subject having cancer.

(67) The use of aspect 66, wherein the additional therapy is selected from the group consisting of surgery, radiation therapy, High Intensity Focused Ultrasound (HIFU), chemotherapy, cryosurgery, hormonal therapy, immunotherapy, targeted monoclonal antibodies, antibody-drug conjugates, tyrosine kinase inhibitors, or a combination thereof.

(68) The use of aspect 66, wherein the additional therapy is an immunotherapy.

(69) The use of any one of aspects 66-68, wherein the additional therapy is an immune checkpoint inhibitor.

(70) The use of any one of aspects 66-69, wherein the additional therapy is an IgG₁ or IgG4 antibody.

(71) The use of aspect 70, wherein the IgG1 or IgG4 antibody is an anti-programmed cell death protein 1 (PD-1) or an anti-programmed death-ligand 1 (PD-L1) antibody.

(72) The use of aspect 71, wherein the antibody is an anti-PD-1 antibody.

(73) The use of aspect 72, wherein the anti-PD-1 antibody comprises a variable light (VL) chain region comprising a CDR1 having the amino acid sequence of SEQ ID NO: 1, a CDR2 having the amino acid sequence of SEQ ID NO: 2, and a CDR3 having the amino acid sequence of SEQ ID NO: 3; and a variable heavy (VH) chain region comprising a CDR1 having the amino acid sequence of SEQ ID NO: 4, a CDR2 having the amino acid sequence of SEQ ID NO: 5, and a CDR3 having the amino acid sequence of SEQ ID NO: 6.

(74) The use of aspect 72, wherein the anti-PD-1 antibody comprises a variable light (VL) chain region comprising a CDR1 having the amino acid sequence of SEQ ID NO: 58, a CDR2 having the amino acid sequence of SEQ ID NO: 59, and a CDR3 having the amino acid sequence of SEQ ID NO: 60; and a variable heavy (VH) chain region comprising a CDR1 having the amino acid sequence of SEQ ID NO: 61, a CDR2 having the amino acid sequence of SEQ ID NO: 62, and a CDR3 having the amino acid sequence of SEQ ID NO: 63.

(75) The use of aspect 72, wherein the anti-PD-1 antibody comprises a variable light (VL) chain region comprising a CDR1 having the amino acid sequence of SEQ ID NO: 64, a CDR2 having the amino acid sequence of SEQ ID NO: 65, and a CDR3 having the amino acid sequence of SEQ ID NO: 66; and a variable heavy (VH) chain region comprising a CDR1 having the amino acid sequence of SEQ ID NO: 67, a CDR2 having the amino acid sequence of SEQ ID NO: 68, and a CDR3 having the amino acid sequence of SEQ ID NO: 69.

(76) The use of aspect 72, wherein the anti-PD-1 antibody comprises a variable light (VL) chain region comprising a CDR1 having the amino acid sequence of SEQ ID NO: 70, a CDR2 having the amino acid sequence of SEQ ID NO: 71, and a CDR3 having the amino acid sequence of SEQ ID NO: 72; and a variable heavy (VH) chain region comprising a CDR1 having the amino acid sequence of SEQ ID NO: 73, a CDR2 having the amino acid sequence of SEQ ID NO: 74, and a CDR3 having the amino acid sequence of SEQ ID NO: 75.

(77) The use of aspect 72, wherein the anti-PD-1 antibody comprises a variable light (VL) chain region comprising a CDR1 having the amino acid sequence of SEQ ID NO: 76, a CDR2 having the amino acid sequence of SEQ ID NO: 77, and a CDR3 having the amino acid sequence of SEQ ID NO: 78; and a variable heavy (VH) chain region comprising a CDR1 having the amino acid sequence of SEQ ID NO: 79, a CDR2 having the amino acid sequence of SEQ ID NO: 80, and a CDR3 having the amino acid sequence of SEQ ID NO: 81.

(78) The use of aspect 71, wherein the antibody is an anti-PD-L1 antibody.

(79) The use of aspect 78, wherein the anti-PD-L1 antibody comprises a variable light (VL) chain region comprising a CDR1 having the amino acid sequence of SEQ ID NO: 7, a CDR2 having the amino acid sequence of SEQ ID NO: 8, and a CDR3 having the amino acid sequence of SEQ ID NO: 9; and a variable heavy (VH) chain region comprising a CDR1 having the amino acid sequence of SEQ ID NO: 10, a CDR2 having the amino acid sequence of SEQ ID NO: 11, and a CDR3 having the amino acid sequence of SEQ ID NO: 12.

(80) The use of aspect 78, wherein the anti-PD-L1 antibody comprises a variable light (VL) chain region comprising a CDR1 having the amino acid sequence of SEQ ID NO: 13, a CDR2 having the amino acid sequence of SEQ ID NO: 14, and a CDR3 having the amino acid sequence of SEQ ID NO: 15; and a variable heavy (VH) chain region comprising a CDR1 having the amino acid sequence of SEQ ID NO: 16, a CDR2 having the amino acid sequence of SEQ ID NO: 17, and a CDR3 having the amino acid sequence of SEQ ID NO: 18.

(81) The use of any one of aspects 71-80, wherein from about 100 mg to about 2,000 mg of the antibody is administered to the subject.

(82) The use of any one of aspects 71-81, wherein the antibody is administered to the subject intravenously.

(83) The use of aspect 82, wherein the antibody is administered to the subject intravenously over about 1 minute to about 60 minutes.

(84) The use of any one of aspects 71-83, wherein the immunoconjugate is concurrently administered with the antibody.

(85) The use of any one of aspects 71-84, wherein the antibody is administered from about every 7 days to about every 35 days.

(86) The use of any one of aspects 60-85, wherein the cancer is a HER2-expressing or HER2-amplified cancer.

(87) The use of any one of aspects 60-86, wherein the cancer is breast cancer.

(88) The use of aspect 87, wherein the cancer is HER2 overexpressing breast cancer.

(89) The use of any one of aspects 60-86, wherein the cancer is gastric cancer.

(90) The use of aspect 89, wherein the cancer is HER2 overexpressing gastric cancer.

(91) The use of any one of aspects 60-86, wherein the cancer is gastroesophageal junction adenocarcinoma.

(92) The use of any one of aspects 60-86, wherein the cancer is colorectal cancer.

(93) The use of any one of aspects 60-86, wherein the cancer is endometrial cancer.

(94) The use of any one of aspects 60-86, wherein the cancer is salivary gland cancer.

(95) The use of any one of aspects 60-86, wherein the cancer is lung cancer.

(96) The use of any one of aspects 60-95, wherein the cancer has metastasized.

(97) The use of any one of aspects 60-96, wherein the cancer is HER2 IHC1+/ISH+.

(98) The use of any one of aspects 60-96, wherein the cancer is HER2 IHC1+/ISH−.

(99) The use of any one of aspects 60-96, wherein the cancer is HER2 IHC2+/ISH+.

(100) The use of any one of aspects 60-96, wherein the cancer is HER2 IHC2+/ISH−.

(101) The use of any one of aspects 60-96, wherein the cancer is HER2 IHC3+.

(102) The use of any one of aspects 60-96, wherein the cancer is expressing or over-expressing HER2 as determined by gene expression.

(103) The use of any one of aspects 60-96, wherein the cancer exhibits HER2 amplification.

(104) The use of aspect 103, wherein the cancer is ISH+.

(105) The use of aspect 103, wherein the cancer is ISH−.

(106) The use of any one of aspects 103-105, wherein the HER2 amplification is determined by sequencing.

(107) The use of any one of aspects 103-105, wherein the HER2 amplification is determined by next generation sequencing (NGS).

(108) The use of any one of aspects 60-107, wherein r is from about 1 to about 6.

(109) The use of aspect 108, wherein r is from about 2 to about 4.

(110) The use of any one of aspects 60-109, wherein n is from about 6 to about 12.

(111) The use of aspect 110, wherein n is about 10.

(112) The use of any one of aspects 60-111, wherein “Ab” is trastuzumab, a biosimilar thereof, or a biobetter thereof.

(113) The use of any one of aspects 60-111, wherein “Ab” is pertuzumab, a biosimilar thereof, or a biobetter thereof.

(114) The use of any one of aspects 60-111, wherein “Ab” is trastuzumab.

(115) The use of any one of aspects 60-111, wherein “Ab” is a biosimilar of trastuzumab.

(116) The use of any one of aspects 60-111, wherein “Ab” is a biosimilar of pertuzumab.

(117) The use of any one of aspects 60-116, wherein the subject is treated for from about 1 month to about 48 months.

(118) The use of any one of aspects 60-117, wherein the subject is human.

### EXAMPLES

The following examples further illustrate the invention but, of course, should not be construed as in any way limiting its scope.

### Example 1

This example demonstrates that BDC-1001 is effective at eliciting myeloid activation and is therefore useful for the treatment of cancer.

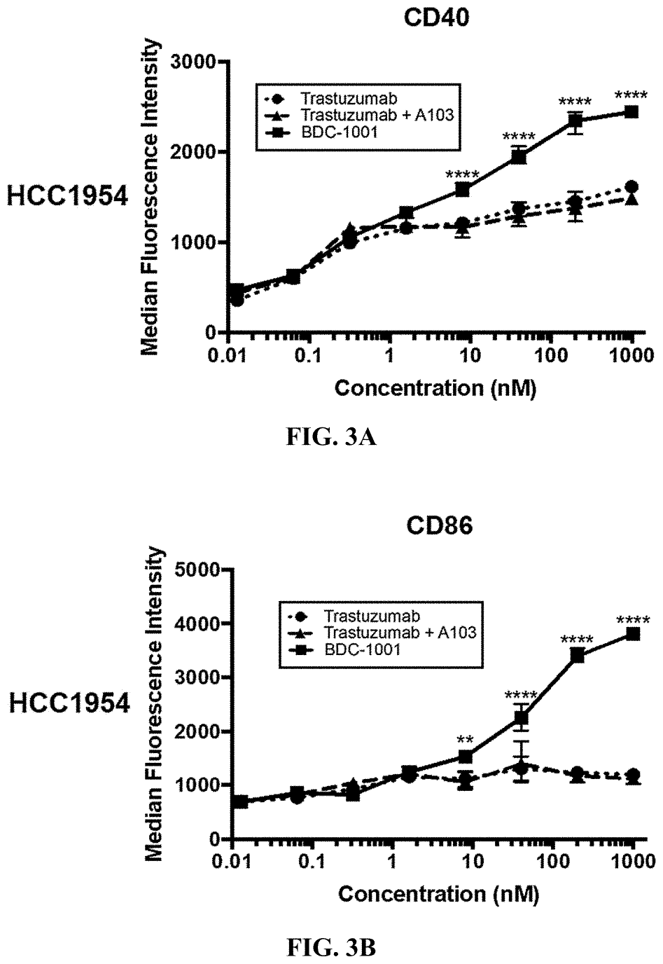

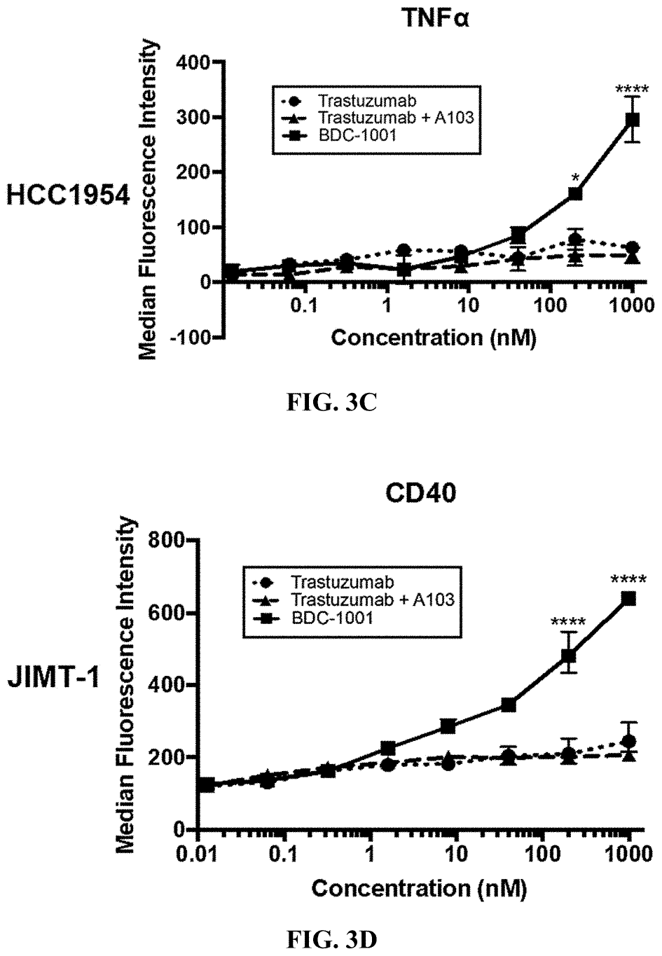

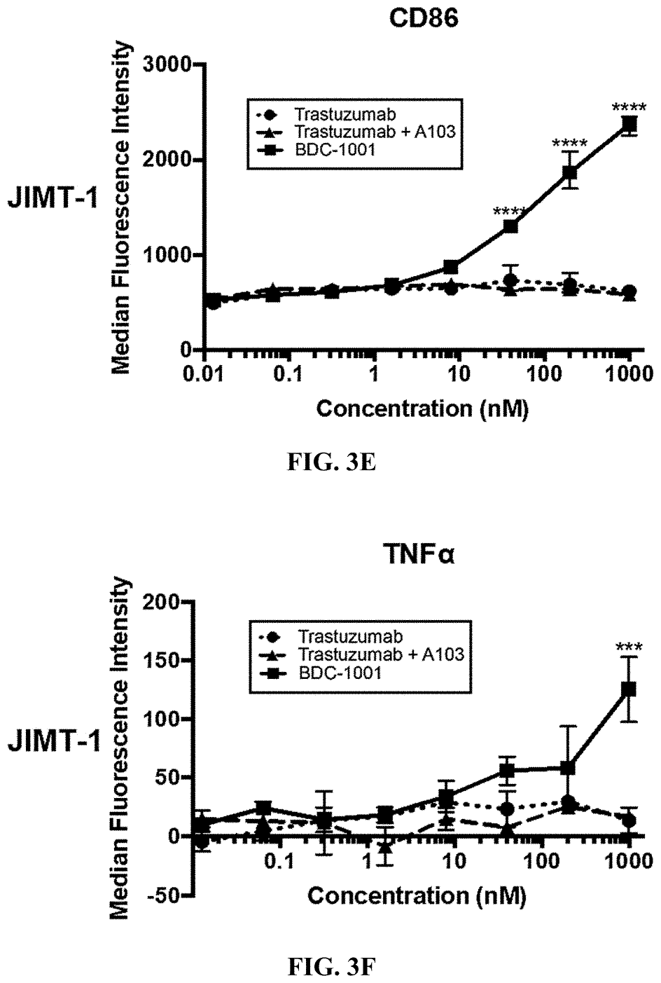

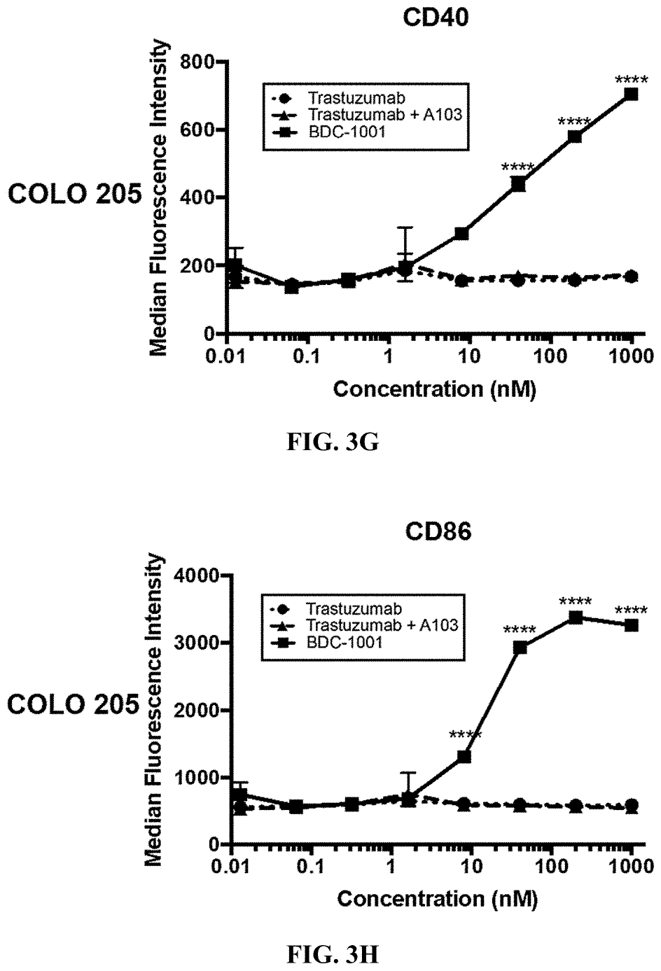

The effect of BDC-1001 on immune activation in myeloid APCs expressing the relevant FcγRs and TLRs was assessed following co-culture of HER2 expressing cancer cell lines with human myeloid APCs at a 10:1 ratio of HER2 expressing cancer cells to human myeloid APCs. After 18 hours, myeloid APCs were analyzed for myeloid activation markers by flow cytometry, and cell free supernatants were analyzed for TNFα secretion. Consistent with TLR7/8 activation, the data presented in FIGS. 3A-3I demonstrate that BDC-1001 elicits enhanced myeloid activation as defined by increased expression of CD40, CD86, and TNFα relative to trastuzumab or the mixture of trastuzumab and the molar equivalent of a conjugate that corresponds to BDC-1001 without trastuzumab (A103). The data presented in FIGS. 3A-3I are from 3 independent experiments and 12 donors (mean and SEM), *P<0.05, **P<0.01, ***P<0.001, ****P<0.0001 as compared across all conditions.

### Example 2

This example demonstrates the favorable primary in vivo pharmacodynamics of BDC-1001.

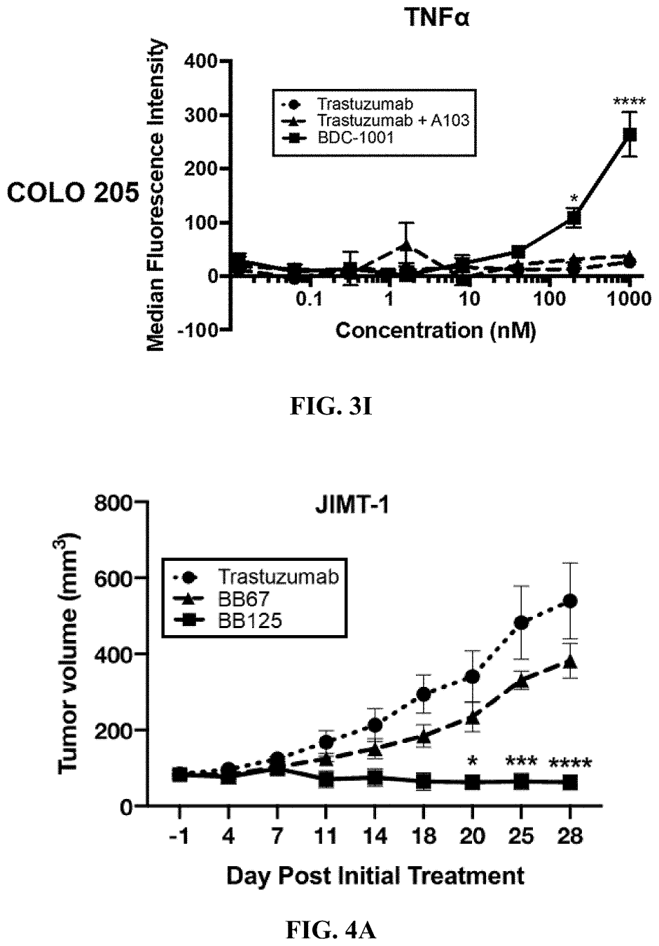

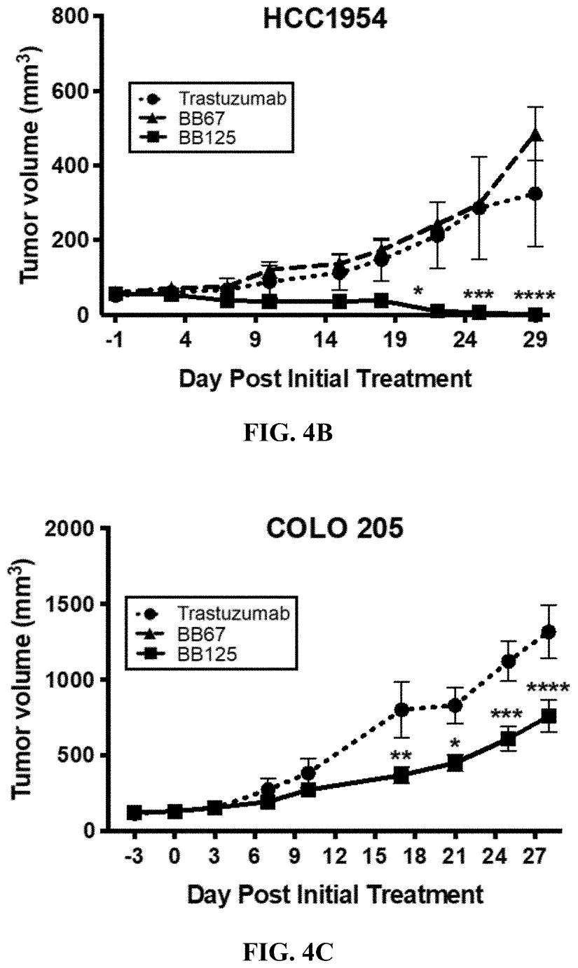

A BDC-1001 surrogate (BB125) was developed that could activate murine myeloid APCs. BDC-1001 has a slightly reduced TNFα EC₅₀ of 281 nM in murine splenocytes. BB125 is a trastuzumab biosimilar (EirGenix, Inc.) covalently attached to a murine TLR7 agonist (CL264, a 9-benzyl-8 hydroxyadenine derivative containing a glycine on the benzyl group, InvivoGen, Inc.) via a non-cleavable (PEG6) linker. Trastuzumab-resistant cell lines that expressed high, intermediate, or low levels of HER2 (HCC1954, JIMT-1, and COLO 205, respectively) were used to assess the capacity of BB125 to mediate anti-tumor activity in vivo in murine models that lack functional B, T, and NK cells (Rag2−/− γc−/− and NSG). These models allowed the growth of human tumor cell lines and enabled the assessment of BB125 on myeloid driven anti-tumor activity. The data presented in FIGS. 4A-4C show that BB125 was significantly more effective at eliciting anti-tumor efficacy than trastuzumab or an isotype control (BB67, which is a rituximab-PEG6-CL264 in trastuzumab-resistant HER2-intermediate and high breast cancer models (JIMT-1 and HCC1954) and HER2-low colorectal tumor model (COLO 205). Greater anti-tumor activity was observed with BB125 in the high (100% tumor growth inhibition (TGI)) and intermediate (88% TGI) HER2 expressing models relative to the low HER2 expressing model (33% TGI). These data indicate that conjugation of trastuzumab to a TLR7 agonist leads to enhanced anti-tumor activity in multiple human xenograft models in mice that retain functional myeloid APCs. Treatment (100 μg, every 5 days×6) was initiated in HCC1954 tumors when they reached an average volume of 50 mm³ whereas treatment was initiated in JIMT-1 and COLO 205 at an average volume of 100 mm³. Rag2−/−γc−/− mice were utilized for HCC1954 and JIMT-1 studies whereas NSG mice were utilized for COLO 205 studies. Percent tumor growth inhibition was calculated relative to trastuzumab. P-values were calculated by two-way ANOVA with Tukey multiple comparisons corrections, where p<0.0001 (****), 0.001 (***), 0.01 (**), 0.05 (*).

### Example 3

This example demonstrates that BDC-1001 has a desirable pharmacokinetic (PK) profile.

A PK assessment was performed in cynomolgus macaques administered 2 doses of BDC-1001 given intravenously 2 weeks apart at 2 dose levels, 10 and 30 mg/kg. The trastuzumab PK assay was configured to capture trastuzumab with HCA169 anti-idiotype mAb and to detect with peroxidase labeled HCA176 (HCA176P). The assay was configured to capture trastuzumab with HCA169 anti-idiotype mAb and to detect with a Sponsor generated rabbit mAb to A103 followed by detection with peroxidase labeled Goat anti-rabbit IgG.

The PK of BDC-1001 was compared to trastuzumab administered intravenously 2 weeks apart at 10 mg/kg. In both instances, the initial dose was administered as an intravenous bolus while the second dose was administered as a 30-minute infusion. The study was designed to allow assessment of the influence of the active species and conjugation on pharmacokinetic parameters. There were no toxicologically significant events observed in this study.

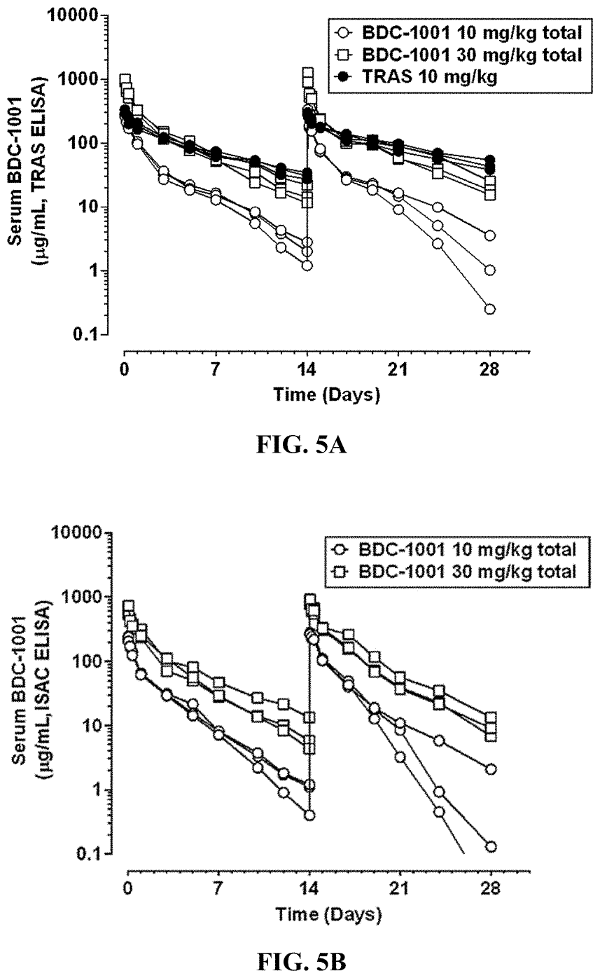

The PK was assessed in separate assays measuring either the quantity of BDC-1001 or the total antibody (“mAb” in Table 1, trastuzumab). BDC-1001 mAb and BDC-1001 demonstrated approximately dose proportional increases in AUC when the dose level increased from 10 to 30 mg/kg. Both the Cmax and t½ of BDC-1001 were lower than trastuzumab at 10 mg/kg. The PK parameters for the second dose were mostly comparable to the PK parameters for the first dose, although trastuzumab and BDC-1001 were administered as a 30-minute infusion rather than a slow bolus (see FIGS. 5A-5B). Animals showing a reduction in trastuzumab and BDC-1001 levels following the second dose were indicative of an anti-active species response.

TABLE 1

| | C_{max (mcg/mL)} | | t_{1/2 (h)} | | AUC _{(h*mcg/mL)} | |
|---|---|---|---|---|---|---|
| | | BDC- | | BDC- | | BDC- |
| Test Article | mAb | 1001 | mAb | 1001 | mAb | 1001 |
| Trastuzumab | 321 | — | 148 | — | 36136 | — |
| 10 mg/kg, Dose 1 | | | | | | |
| BDC-1001 | 277 | 50 | 63 | 50 | 10698 | 7626 |
| 10 mg/kg, Dose 1 | | | | | | |
| BDC-1001 | 956 | 73 | 86 | 73 | 40249 | 27582 |
| 30 mg/kg, Dose 1 | | | | | | |
| Trastuzumab | 291 | — | 179 | — | 45108 | — |
| 10 mg/kg, Dose 2 | | | | | | |
| BDC-1001 | 321 | 39 | 54 | 39 | 9224 | 10780 |
| 10 mg/kg, Dose 2 | | | | | | |
| BDC-1001 | 1081 | 64 | 93 | 64 | 37606 | 40101 |
| 30 mg/kg, Dose 2 | | | | | | |

For intravenous (IV) bolus or short (15 minute) antibody or BDC-1001 infusion (Dose 1), Co was extrapolated using the first 2 time points. Cₘₐₓ was the maximum measured or extrapolated serum concentration. For long (30 minute) infusions (Dose 2), Cₘₐₓ is the maximum measured serum concentration. Antibody and BDC-1001 half-life values were determined from the terminal elimination rate constant (kₑₗ) using the last 5 time points for each dose. AUC (AUC_{0-inf}) was integrated to infinity.

### Example 4

This example demonstrates that cynomolgus monkeys were an appropriate species for toxicological investigation of BDC-1001.

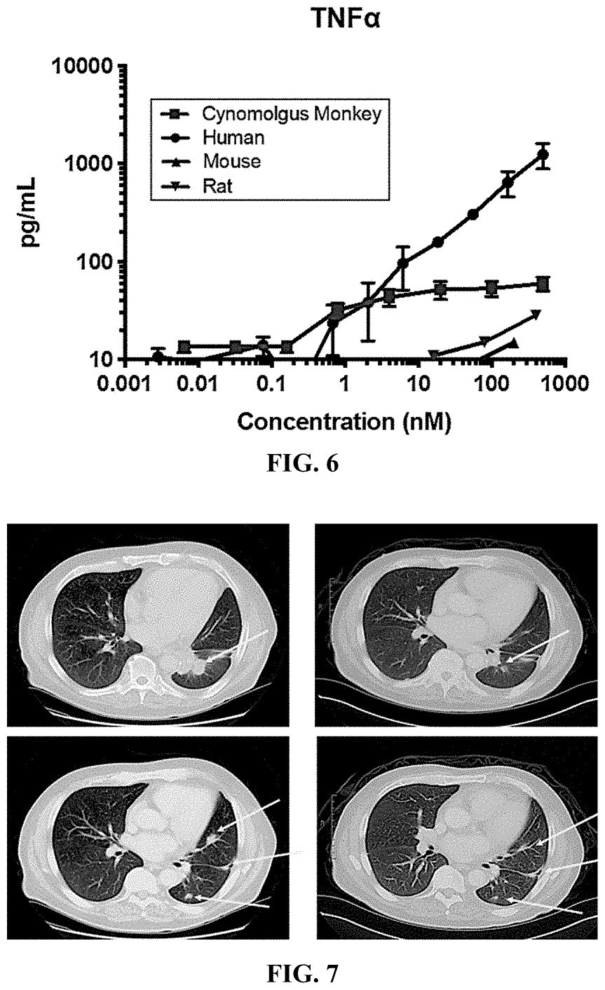

Splenocytes (mouse, rat) or PBMCs (human, cynomolgus monkey) were incubated with BDC 1001 in the absence of HER2 expressing target cells and assayed for TNFα secretion. While the amplitude of the response was different, the data presented in FIG. 6 indicates that human and cynomolgus monkey PBMCs responded to BDC-1001 at concentrations of 1 nM and above whereas rat and mouse splenocytes were generally unresponsive. Rat and mouse peripheral blood leukocytes (PBL) showed similar results as splenocytes.

### Example 5

This example demonstrates two of the potential strategies in which BDC-1001 can be used to treat human tumors: (1) used as a monotherapy and (2) used in combination with an immune checkpoint inhibitor (e.g., pembrolizumab and nivolumab) in subjects with advanced solid tumors, including subjects with advanced HER2 expressing or HER2-amplified solid tumors.

The therapy scheme is as shown in FIG. 1. For monotherapy, the exclusion criteria include the following: (a) a history of treatment with a TLR7, TLR8, or a TLR7/8 agonist, (b) use of another investigational agent or anticancer therapy within 4 weeks prior to CID1 or within 5 estimated elimination half-lives, whichever is shorter, (c) use of another anti-HER2 based therapy within 4 weeks prior to CID1, and (d) history of severe hypersensitivity to any ingredient of the study, including trastuzumab. For anti-PD1 combination therapy, the exclusion criteria include the following: (a) history of immune-mediated colitis, (b) an active autoimmune disease, with the exception of autoimmune endocrinopathies, that is stable on hormone replacement therapy, and (c) hypersensitivity to pembrolizumab or any of the excipients used in the formulation.

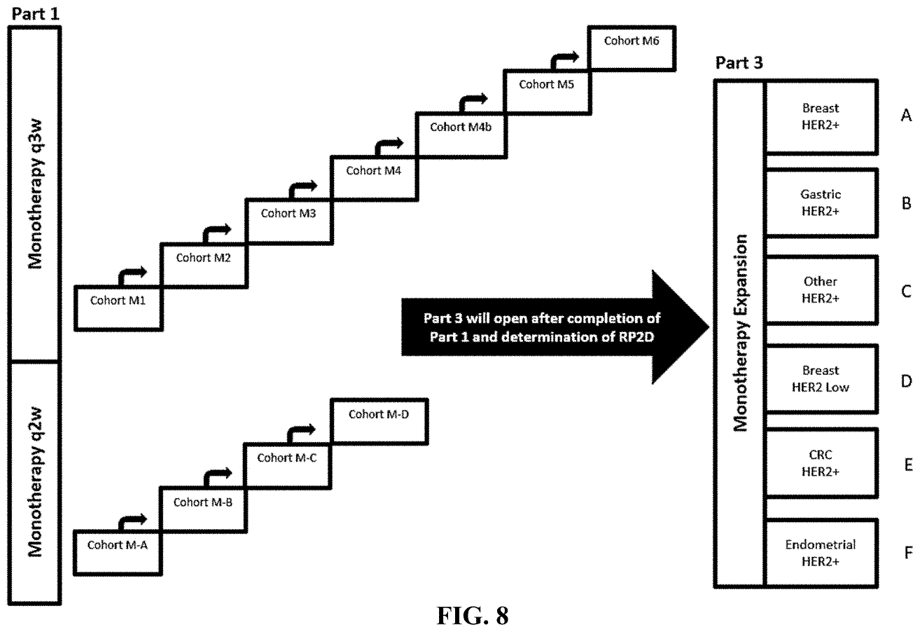

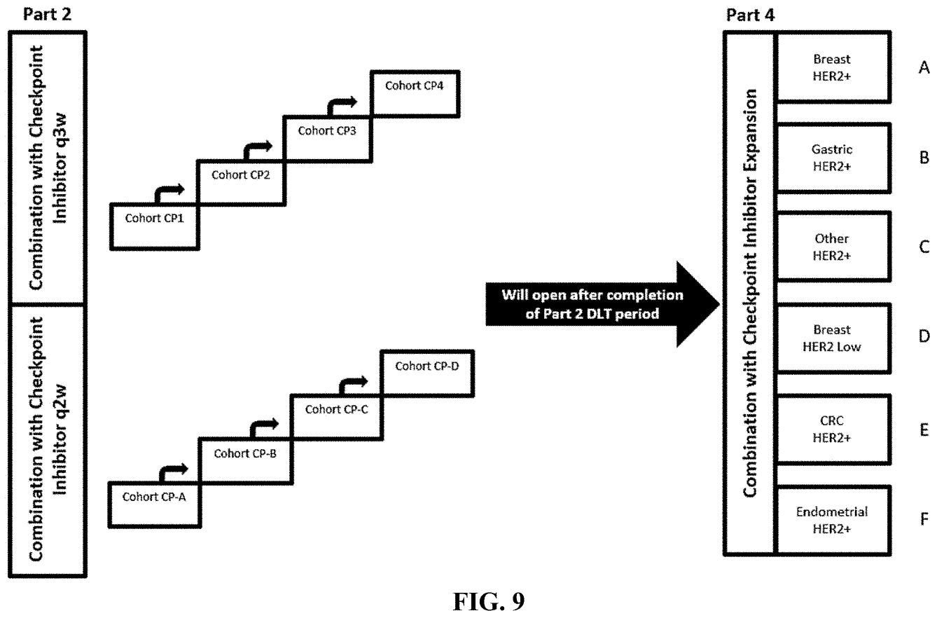

Additional therapy schemes involve dosing BDC-1001 as monotherapy at 0.15, 0.5, 2, 5, 8, 12, and 20 mg/kg every 2 weeks or every 3 weeks by IV infusion (see FIG. 8). A further therapy scheme involves dosing BDC-1001 as monotherapy at 0.15, 0.5, 2, 5, 8, 12, and 20 mg/kg every 2 weeks or every 3 weeks by IV infusion in combination with an immune checkpoint inhibitor, such as pembrolizumab or nivolumab (see FIG. 9).

Preliminary data: As of Jan. 29, 2021, 20 patients were enrolled in the study across four cohorts at escalating dose levels. The lowest dose cohort of 0.15 mg/kg required a single patient to assess tolerability to proceed to the next dose level. Each subsequent cohort enrolled an initial three patients to evaluate for dose-limiting toxicities, after which an additional 12 patients were able to be enrolled to the cohorts and escalate to the next dose level if the safety criteria were met. One patient was enrolled in the 0.15 mg/kg cohort, and three patients were enrolled in the 0.5 mg/kg cohort. These dose levels were well tolerated by all four patients, who completed the safety evaluation period without incident. Neither dose was expected to be therapeutically active based on preclinical modeling. Four patients were enrolled, which included one additional patient, in the 2 mg/kg cohort. Twelve patients were enrolled in the 5 mg/kg cohort. In the 2 mg/kg and 5 mg/kg cohorts, early signs of clinical activity as well as changes in pharmacodynamic biomarkers were observed that appear to be consistent with the proposed mechanism of action.

In the 2 mg/kg cohort, four patients were enrolled with the following cancers: biliary, gastric, rectal and uterine. These patients remained on study with treatment duration ranging from five weeks to 15 weeks. One unconfirmed stable disease was observed in the patient with rectal cancer, who remained on study for 11 weeks. Confirmed stable disease in the patient was observed with microsatellite-stable uterine cancer with visceral lung metastases. This patient remains on treatment and has received six doses of BDC-1001 and is in her 17th week of treatment.

In the 5 mg/kg cohort, we have enrolled 12 patients as of Jan. 29, 2021, with the following cancers: cervix, uterine, colon, esophageal, GE junction, rectal, lung, salivary ductal, and bladder. Five patients remained on study at this dose level, with treatment durations ranging from two weeks to 12 weeks. Unconfirmed stable disease in two patients was observed with colorectal cancer, all of whom have visceral lung or both lung and liver metastases. Each of these patients remained on study and had their first CT scan at six weeks, i.e., after two doses of BDC-1001. The first CT scan for one of these patients (66 year old male with progressive adenocarcinoma of the colon, metastatic to lungs, and with microsatellite-stable colorectal cancer) demonstrated a 36% reduction in tumor size of lung target lesions based upon RECIST 1.1 criteria. The second CT scan of this patient at 12 weeks demonstrated a 39% reduction in the sum of the longest diameters of all four measurable tumor lesions, and qualified as a confirmed partial response based upon RECIST 1.1 criteria (see FIG. 7). The images on the left of FIG. 7 were taken before BDC-1001 treatment and the images on the right were taken after 2 cycles of BDC-1001 treatment. The top images of FIG. 7 are from a first section and the bottom images of FIG. 7 are from a second section. The arrows in the images of FIG. 7 are pointing to the tumor lesions. As of Jan. 29, 2021, this patient remains on treatment and has received four doses of BDC-1001 and is in his 12th week of treatment. Prior to enrollment, this patient had tumor progression despite multiple prior therapies, including chemotherapy, anti-angiogenesis, and PD-1 inhibitor administration.

BDC-1001 has been well tolerated to date in all 20 patients. All subjects have completed their 21-day DLT evaluation period (excluding the 20th patient who was recently enrolled and is still in the DLT period), and no DLTs or drug-related serious adverse events have been observed. Treatment-emergent adverse events deemed to be related to BDC-1001 have been mild or moderate in severity, including mild infusion-related reactions without interruption to dosing. Patients are continuously enrolled in the study including open enrollment in the next higher dose level cohort at 8 mg/kg.

In addition to the clinical observations, elevations in pharmacodynamic markers such as plasma cytokines and chemokines were observed with a trend towards greater magnitude in patients with increasing dose level. The elevations in pharmacodynamic markers include increases in plasma levels of MCP-1, MIP1α, and IP-10, which are chemokines consistent with myeloid cell activation. Transient increases in plasma levels of TNFα, an indicator of TLR activation also have been observed. The plasma cytokine and chemokine data are consistent with the preclinical data and also appear to be consistent with the proposed mechanism of action of BDC-1001.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for treating cancer in a subject comprising administering from about 0.01 to about 100 mg/kg of an immunoconjugate of formula: Ab-[TA]ᵣ or a pharmaceutically acceptable salt thereof, wherein “Ab” is an antibody construct that has an antigen binding domain that binds human epidermal growth factor receptor type 2 (HER2) and “TA” is a therapeutic agent of formula: 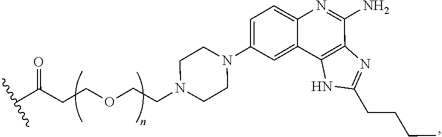
wherein n is from about 2 to about 25 and r is an average therapeutic agent to antibody ratio from about 1 to about 10, to a subject having cancer.

2. A method for treating cancer in a subject comprising administering from about every 3 to about every 45 days an immunoconjugate of formula: Ab-[TA]ᵣ or a pharmaceutically acceptable salt thereof, wherein “Ab” is an antibody construct that has an antigen binding domain that binds human epidermal growth factor receptor type 2 (HER2) and “TA” is a therapeutic agent of formula: 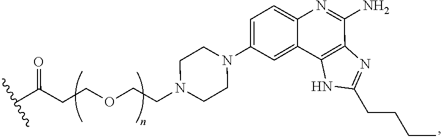
wherein n is from about 2 to about 25 and r is an average therapeutic agent to antibody ratio from 1 to 10, to a subject having cancer.

3. The method of claim 2, wherein from about 0.01 to about 100 mg/kg of an immunoconjugate is administered to the subject during each administration.

4. The method of claim 1, wherein the immunoconjugate is administered in the form of a composition comprising the immunoconjugate and a pharmaceutically acceptable carrier therefor.

5. The method of claim 1, T wherein the immunoconjugate is administered to the subject intravenously.

6. The method of claim 5, wherein the immunoconjugate is administered to the subject intravenously over about 1 to about 240 minutes.

7. The method of claim 1, further comprising administering an effective amount of an additional therapy to the subject having cancer.

8. The method of claim 7, wherein the additional therapy is selected from the group consisting of surgery, radiation therapy, High Intensity Focused Ultrasound (HIFU), chemotherapy, cryosurgery, hormonal therapy, immunotherapy, targeted monoclonal antibodies, antibody-drug conjugates, tyrosine kinase inhibitors, or a combination thereof.

9. The method of claim 7, wherein the additional therapy is an immunotherapy.

10. (canceled)

11. (canceled)

12. The method of claim 9, wherein the additional therapy is an anti-programmed cell death protein 1 (PD-1) or an anti-programmed death-ligand 1 (PD-L1) antibody.

13-26. (canceled)

27. The method of claim 1, wherein the cancer is a HER2-expressing or HER2-amplified cancer.

28.-37. (canceled)

38. The method of claim 1, wherein the cancer is HER2 IHC1+/ISH+.

39. The method of claim 1, wherein the cancer is HER2 IHC1+/ISH−.

40. The method of claim 1, wherein the cancer is HER2 IHC2+/ISH+.

41. The method of claim 1, wherein the cancer is HER2 IHC2+/ISH−.

42. The method of claim 1, wherein the cancer is HER2 IHC3+.

43.-52. (canceled)

53. The method of claim 1, wherein “Ab” is trastuzumab, a biosimilar thereof, or a biobetter thereof.

54. The method of claim 1, wherein “Ab” is pertuzumab, a biosimilar thereof, or a biobetter thereof.

55.-57. (canceled)

58. The method of claim 1, wherein the subject is treated for from about 1 month to about 48 months.

59. The method of claim 1, wherein the subject is human.


SEQUENCE LISTING (117 sequences)

SEQ ID NO: 1 - protein, 15 aa, Artificial Sequence
  Synthetic
RASKGVSTSGYSYLH

SEQ ID NO: 2 - protein, 7 aa, Artificial Sequence
  Synthetic
LASYLES

SEQ ID NO: 3 - protein, 9 aa, Artificial Sequence
  Synthetic
QHSRDLPLT

SEQ ID NO: 4 - protein, 5 aa, Artificial Sequence
  Synthetic
NYYMY

SEQ ID NO: 5 - protein, 16 aa, Artificial Sequence
  Synthetic
GINPSNGGTNFNEKFK

SEQ ID NO: 6 - protein, 11 aa, Artificial Sequence
  Synthetic
RDYRFDMGFDY

SEQ ID NO: 7 - protein, 11 aa, Artificial Sequence
  Synthetic
RASQDVSTAVA

SEQ ID NO: 8 - protein, 7 aa, Artificial Sequence
  Synthetic
SASFLYS

SEQ ID NO: 9 - protein, 9 aa, Artificial Sequence
  Synthetic
QQYLYHPAT

SEQ ID NO: 10 - protein, 5 aa, Artificial Sequence
  Synthetic
DSWIH

SEQ ID NO: 11 - protein, 17 aa, Artificial Sequence
  Synthetic
WISPYGGSTYYADSVKG

SEQ ID NO: 12 - protein, 9 aa, Artificial Sequence
  Synthetic
RHWPGGFDY

SEQ ID NO: 13 - protein, 14 aa, Artificial Sequence
  Synthetic
TGTSSDVGGYNYVS

SEQ ID NO: 14 - protein, 7 aa, Artificial Sequence
  Synthetic
DVSNRPS

SEQ ID NO: 15 - protein, 10 aa, Artificial Sequence
  Synthetic
SSYTSSSTRV

SEQ ID NO: 16 - protein, 5 aa, Artificial Sequence
  Synthetic
SYIMM

SEQ ID NO: 17 - protein, 17 aa, Artificial Sequence
  Synthetic
SIYPSGGITFYADTVKG

SEQ ID NO: 18 - protein, 11 aa, Artificial Sequence
  Synthetic
IKLGTVTTVDY

SEQ ID NO: 19 - protein, 1255 aa, Artificial Sequence [sequence omitted]

SEQ ID NO: 20 - protein, 11 aa, Artificial Sequence
  Synthetic
RASQDVNTAVA

SEQ ID NO: 21 - protein, 7 aa, Artificial Sequence
  Synthetic
SASFLYS

SEQ ID NO: 22 - protein, 9 aa, Artificial Sequence
  Synthetic
QQHYTTPPT

SEQ ID NO: 23 - protein, 5 aa, Artificial Sequence
  Synthetic
DTYIH

SEQ ID NO: 24 - protein, 17 aa, Artificial Sequence
  Synthetic
RIYPTNGYTRYADSVKG

SEQ ID NO: 25 - protein, 11 aa, Artificial Sequence
  Synthetic
WGGDGFYAMDY

SEQ ID NO: 26 - protein, 23 aa, Artificial Sequence
  Synthetic
DIQMTQSPSSLSASVGDRVTITC

SEQ ID NO: 27 - protein, 15 aa, Artificial Sequence
  Synthetic
WYQQKPGKAPKLLIY

SEQ ID NO: 28 - protein, 32 aa, Artificial Sequence
  Synthetic
GVPSRFSGSRSGTDFTLTISSLQPEDFATYYC

SEQ ID NO: 29 - protein, 10 aa, Artificial Sequence
  Synthetic
FGQGTKVEIK

SEQ ID NO: 30 - protein, 30 aa, Artificial Sequence
  Synthetic
EVQLVESGGGLVQPGGSLRLSCAASGFNIK

SEQ ID NO: 31 - protein, 14 aa, Artificial Sequence
  Synthetic
WVRQAPGKGLEWVA

SEQ ID NO: 32 - protein, 32 aa, Artificial Sequence
  Synthetic
RFTISADTSKNTAYLQMNSLRAEDTAVYYCSR

SEQ ID NO: 33 - protein, 11 aa, Artificial Sequence
  Synthetic
WGQGTLVTVSS

SEQ ID NO: 34 - protein, 11 aa, Artificial Sequence
  Synthetic
KASQDVSIGVA

SEQ ID NO: 35 - protein, 7 aa, Artificial Sequence
  Synthetic
SASYRYT

SEQ ID NO: 36 - protein, 9 aa, Artificial Sequence
  Synthetic
QQYYIYPYT

SEQ ID NO: 37 - protein, 5 aa, Artificial Sequence
  Synthetic
DYTMD

SEQ ID NO: 38 - protein, 17 aa, Artificial Sequence
  Synthetic
DVNPNSGGSIYNQRFKG

SEQ ID NO: 39 - protein, 10 aa, Artificial Sequence
  Synthetic
NLGPSFYFDY

SEQ ID NO: 40 - protein, 23 aa, Artificial Sequence
  Synthetic
DIQMTQSPSSLSASVGDRVTITC

SEQ ID NO: 41 - protein, 15 aa, Artificial Sequence
  Synthetic
WYQQKPGKAPKLLIY

SEQ ID NO: 42 - protein, 32 aa, Artificial Sequence
  Synthetic
GVPSRFSGSGSGTDFTLTISSLQPEDFATYYC

SEQ ID NO: 43 - protein, 10 aa, Artificial Sequence
  Synthetic
FGQGTKVEIK

SEQ ID NO: 44 - protein, 30 aa, Artificial Sequence
  Synthetic
EVQLVESGGGLVQPGGSLRLSCAASGFTFT

SEQ ID NO: 45 - protein, 14 aa, Artificial Sequence
  Synthetic
WVRQAPGKGLEWVA

SEQ ID NO: 46 - protein, 32 aa, Artificial Sequence
  Synthetic
RFTLSVDRSKNTLYLQMNSLRAEDTAVYYCAR

SEQ ID NO: 47 - protein, 11 aa, Artificial Sequence
  Synthetic
WGQGTLVTVSS

SEQ ID NO: 48 - protein, 214 aa, Artificial Sequence
  Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 450 aa, Artificial Sequence
  Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 214 aa, Artificial Sequence
  Synthetic
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 448 aa, Artificial Sequence
  Synthetic
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 12 aa, Artificial Sequence
  Synthetic
RASQRVSSSYLA

SEQ ID NO: 53 - protein, 7 aa, Artificial Sequence
  Synthetic
DASSRAT

SEQ ID NO: 54 - protein, 9 aa, Artificial Sequence
  Synthetic
QQYGSLPWT

SEQ ID NO: 55 - protein, 5 aa, Artificial Sequence
  Synthetic
RYWMS

SEQ ID NO: 56 - protein, 17 aa, Artificial Sequence
  Synthetic
NIKQDGSEKYYVDSVKG

SEQ ID NO: 57 - protein, 12 aa, Artificial Sequence
  Synthetic
EGGWFGELAFDY

SEQ ID NO: 58 - protein, 11 aa, Artificial Sequence
  Synthetic
RASQSVSSYLA

SEQ ID NO: 59 - protein, 7 aa, Artificial Sequence
  Synthetic
DASNRAT

SEQ ID NO: 60 - protein, 9 aa, Artificial Sequence
  Synthetic
QQSSNWPRT

SEQ ID NO: 61 - protein, 5 aa, Artificial Sequence
  Synthetic
NSGMH

SEQ ID NO: 62 - protein, 17 aa, Artificial Sequence
  Synthetic
VIWYDGSKRYYADSVKG

SEQ ID NO: 63 - protein, 4 aa, Artificial Sequence
  Synthetic
NDDY

SEQ ID NO: 64 - protein, 11 aa, Artificial Sequence
  Synthetic
KSSESVSNDVA

SEQ ID NO: 65 - protein, 7 aa, Artificial Sequence
  Synthetic
YAFHRFT

SEQ ID NO: 66 - protein, 9 aa, Artificial Sequence
  Synthetic
HQAYSSPYT

SEQ ID NO: 67 - protein, 5 aa, Artificial Sequence
  Synthetic
SYGVH

SEQ ID NO: 68 - protein, 16 aa, Artificial Sequence
  Synthetic
VIYADGSTNYNPSLKS

SEQ ID NO: 69 - protein, 10 aa, Artificial Sequence
  Synthetic
AYGNYWYIDV

SEQ ID NO: 70 - protein, 11 aa, Artificial Sequence
  Synthetic
KASQDVGTAVA

SEQ ID NO: 71 - protein, 7 aa, Artificial Sequence
  Synthetic
WASTLHT

SEQ ID NO: 72 - protein, 9 aa, Artificial Sequence
  Synthetic
QHYSSYPWT

SEQ ID NO: 73 - protein, 5 aa, Artificial Sequence
  Synthetic
SYDMS

SEQ ID NO: 74 - protein, 17 aa, Artificial Sequence
  Synthetic
TISGGGSYTYYQDSVKG

SEQ ID NO: 75 - protein, 7 aa, Artificial Sequence
  Synthetic
PYYAMDY

SEQ ID NO: 76 - protein, 14 aa, Artificial Sequence
  Synthetic
TGTSSDVGFYNYVS

SEQ ID NO: 77 - protein, 7 aa, Artificial Sequence
  Synthetic
DVSNRPS

SEQ ID NO: 78 - protein, 10 aa, Artificial Sequence
  Synthetic
SSYTSISTWV

SEQ ID NO: 79 - protein, 7 aa, Artificial Sequence
  Synthetic
STTYYWV

SEQ ID NO: 80 - protein, 16 aa, Artificial Sequence
  Synthetic
SISYSGSTYYNPSLKS

SEQ ID NO: 81 - protein, 13 aa, Artificial Sequence
  Synthetic
HLGYNGRYLPFDY

SEQ ID NO: 82 - protein, 23 aa, Artificial Sequence
  Synthetic
EIVLTQSPATLSLSPGERATLSC

SEQ ID NO: 83 - protein, 15 aa, Artificial Sequence
  Synthetic
WYQQKPGQAPRLLIY

SEQ ID NO: 84 - protein, 32 aa, Artificial Sequence
  Synthetic
GIPARFSGSGSGTDFTLTISSLEPEDFAVYYC

SEQ ID NO: 85 - protein, 10 aa, Artificial Sequence
  Synthetic
FGQGTKVEIK

SEQ ID NO: 86 - protein, 30 aa, Artificial Sequence
  Synthetic
QVQLVESGGGVVQPGRSLRLDCKASGITFS

SEQ ID NO: 87 - protein, 14 aa, Artificial Sequence
  Synthetic
WVRQAPGKGLEWVA

SEQ ID NO: 88 - protein, 32 aa, Artificial Sequence
  Synthetic
RFTISRDNSKNTLFLQMNSLRAEDTAVYYCAT

SEQ ID NO: 89 - protein, 11 aa, Artificial Sequence
  Synthetic
WGQGTLVTVSS

SEQ ID NO: 90 - protein, 23 aa, Artificial Sequence
  Synthetic
DIVMTQSPDSLAVSLGERATINC

SEQ ID NO: 91 - protein, 15 aa, Artificial Sequence
  Synthetic
WYQQKPGQPPKLLIN

SEQ ID NO: 92 - protein, 32 aa, Artificial Sequence
  Synthetic
GVPDRFSGSGYGTDFTLTISSLQAEDVAVYYC

SEQ ID NO: 93 - protein, 10 aa, Artificial Sequence
  Synthetic
FGQGTKLEIK

SEQ ID NO: 94 - protein, 30 aa, Artificial Sequence
  Synthetic
QVQLQESGPGLVKPSETLSLTCTVSGFSLT

SEQ ID NO: 95 - protein, 14 aa, Artificial Sequence
  Synthetic
WIRQPPGKGLEWIG

SEQ ID NO: 96 - protein, 32 aa, Artificial Sequence
  Synthetic
RVTISKDTSKNQVSLKLSSVTAADTAVYYCAR

SEQ ID NO: 97 - protein, 11 aa, Artificial Sequence
  Synthetic
WGQGTTVTVSS

SEQ ID NO: 98 - protein, 23 aa, Artificial Sequence
  Synthetic
DIQLTQSPSFLSAYVGDRVTITC

SEQ ID NO: 99 - protein, 15 aa, Artificial Sequence
  Synthetic
WYQQKPGKAPKLLIY

SEQ ID NO: 100 - protein, 32 aa, Artificial Sequence
  Synthetic
GVPSRFSGSGSGTEFTLTISSLQPEDFATYYC

SEQ ID NO: 101 - protein, 30 aa, Artificial Sequence
  Synthetic
EVQLLESGGGLVQPGGSLRLSCAASGFTFS

SEQ ID NO: 102 - protein, 14 aa, Artificial Sequence
  Synthetic
WVRQAPGKGLEWVS

SEQ ID NO: 103 - protein, 32 aa, Artificial Sequence
  Synthetic
RFTISRDNSKNTLYLQMNSLRAEDTAVYYCAS

SEQ ID NO: 104 - protein, 22 aa, Artificial Sequence
  Synthetic
QSALTQPASVSGSPGQSITISC

SEQ ID NO: 105 - protein, 15 aa, Artificial Sequence
  Synthetic
WYQQHPGKAPELMIY

SEQ ID NO: 106 - protein, 32 aa, Artificial Sequence
  Synthetic
GVSDRFSGSKSGNTASLTISGLQAEDEADYYC

SEQ ID NO: 107 - protein, 10 aa, Artificial Sequence
  Synthetic
FGGGTKLTVL

SEQ ID NO: 108 - protein, 30 aa, Artificial Sequence
  Synthetic
QLQLQESGPGLVKPSETLTLTCTVSADSIS

SEQ ID NO: 109 - protein, 32 aa, Artificial Sequence
  Synthetic
RVTVSVDTSKNQFSLKLNSVAATDTALYYCAR

SEQ ID NO: 110 - protein, 107 aa, Artificial Sequence [sequence omitted]

SEQ ID NO: 111 - protein, 113 aa, Artificial Sequence [sequence omitted]

SEQ ID NO: 112 - protein, 107 aa, Artificial Sequence
  Synthetic
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 113 - protein, 118 aa, Artificial Sequence
  Synthetic
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 114 - protein, 107 aa, Artificial Sequence
  Synthetic
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIK

SEQ ID NO: 115 - protein, 116 aa, Artificial Sequence
  Synthetic
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSS

SEQ ID NO: 116 - protein, 110 aa, Artificial Sequence
  Synthetic
QSALTQPASVSGSPGQSITISCTGTSSDVGFYNYVSWYQQHPGKAPELMIYDVSNRPSGVSDRFSGSKSGNTASLTISGLQAEDEADYYCSSYTSISTWVFGGGTKLTVL

SEQ ID NO: 117 - protein, 123 aa, Artificial Sequence
  Synthetic
QLQLQESGPGLVKPSETLTLTCTVSADSISSTTYYWVWIRQPPGKGLEWIGSISYSGSTYYNPSLKSRVTVSVDTSKNQFSLKLNSVAATDTALYYCARHLGYNGRYLPFDYWGQGTLVTVSS